(19) United States
(12) Patent Application Publication
(10) Pub. No.: US 2020/0392084 A1
(43) Pub. Date: Dec. 17, 2020

(54) SULFASALAZINE SALT COMPOSITIONS AND METHODS OF USING THE SAME

(71) Applicants: Khawla ABU-IZZA, Emeryville, CA (US); David PEARSON, Edinburgh (GB); Lorna KENNEDY, Edinburgh (GB); Joseph BENSON, Edinburgh (GB)

(72) Inventors: Khawla ABU-IZZA, Emeryville, CA (US); David PEARSON, Edinburgh (GB); Lorna KENNEDY, Edinburgh (GB); Joseph BENSON, Edinburgh (GB)

(21) Appl. No.: 16/753,971

(22) PCT Filed: Oct. 9, 2018
(86) PCT No.: PCT/US2018/054983

§ 371 Date: Apr. 6, 2020

Related U.S. Application Data
(60) Provisional application No. 62/570,258, filed on Oct. 10, 2017.

(51) Int. Cl. C07D 213/76; A61K 45/06; A61K 31/655

(52) U.S. Cl. CPC C07D 213/76; C07B 2200/13; A61K 31/655; A61K 45/06

(57) ABSTRACT

Sulfasalazine salt compositions are provided. In some cases, the sulfasalazine salts have a crystalline form. The subject crystalline sulfasalazine salts can provide a water soluble form of the active compound that finds use in pharmaceutical compositions and therapeutic applications. The subject crystalline sulfasalazine salts can provide increased solubility as compared to the zwitterionic or free acid form of sulfasalazine. Also provided are pharmaceutical compositions including the subject sulfasalazine salt compositions. Methods of treating a neurological related disease such as refractory epilepsy using the subject crystalline sulfasalazine salts and pharmaceutical compositions are also provided.

### CROSS-REFERENCE

This application is a 371 National Phase of International Application Serial No. PCT/US2018/054983 filed Oct. 9, 2018, which application claims the benefit of priority to U.S. Provisional Patent Application No. 62/570,258, filed Oct. 10, 2017, which applications are incorporated herein by reference in their entirety.

### INTRODUCTION

Sulfasalazine was synthesized to combine an antibiotic, sulfapyridine, and an anti-inflammatory agent, 5-aminosalicylic acid (5-ASA). Sulfasalazine finds use in treatment of rheumatoid arthritis and inflammatory bowel diseases including ulcerative colitis and Crohn's disease. Sulfasalazine can be metabolized in vivo to sulfapyridine and 5-ASA. Significant side effects occur in about 25% of people. Commonly these include loss of appetite, nausea, headache, and rash. Severe side effects include bone marrow suppression, liver problems, and kidney problems.

Sulfasalazine is a poorly soluble drug when in a free acid form. The presence of azo and sulfonamide linkages in the chemical structure of the drug also makes sulfasalazine prone to degradation during the various stages of formulation manufacturing, leading to the probable appearance of degradation-related impurities in the final product. While the sulfonamide linkage is susceptible to hydrolysis in acidic medium to form the corresponding sulfonic acid derivative and amine, the azo group can undergo chemical changes under hydrolytic, photolytic, and oxidative conditions to form different degradation products. Based on these chemical susceptibilities, there are several possible degradation products of sulfasalazine (Saini et al. “Degradation Study on Sulfasalazine and a Validated HPLC-UV Method for its Stability Testing”, Sci Pharm. 2014; 82: 295-306).

Pharmaceutical solids can exist in different crystal forms, such as crystalline, amorphous, or glass and also in solvated or hydrated states. Polymorphism is the ability of any element or compound to crystallize as more than one distinct crystal species. Different polymorphic forms of the same drug may have substantial differences in certain pharmaceutically-important physicochemical properties, such as stability, solubility, dissolution rate, crystal habit, tableting behavior. Changes in certain of these physiochemical properties may affect the bioavailability of the drug. Forms of sulfasalazine suitable for use in development of pharmaceutical compositions for use in treatment of disease are of interest.

### SUMMARY

Sulfasalazine salt compositions are provided. In some cases, the sulfasalazine salts have a crystalline form. The subject crystalline sulfasalazine salts can provide a water-soluble form of the active compound that finds use in pharmaceutical compositions and therapeutic applications. The subject crystalline sulfasalazine salts can provide increased solubility as compared to the zwitterionic or free acid form of sulfasalazine. The subject crystalline sulfasalazine salts can also, in some cases, provide increased stability of the active compound in a composition that finds use in a variety of therapeutic applications. As such, also provided are pharmaceutical compositions including the subject sulfasalazine salt compositions. Methods of treating a neurological related disease such as refractory epilepsy using the subject crystalline sulfasalazine salts and pharmaceutical compositions are also provided.

These and other objects, advantages, and features of the present invention will become apparent to those persons skilled in the art upon reading the details of the sulfasalazine salt compositions and methods of using the same as more fully described below.

### BRIEF DESCRIPTION OF THE DRAWINGS

The present disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 shows the X-ray Powder Diffraction (XRPD) diffractograms of small scale (top panel) and scaled up (bottom panel) preparations of a benzenesulfonic acid salt of sulfasalazine.

FIG. 2 shows Dynamic Vapor Sorption (DVS) isotherm plots of cycles or sorption/desorption of a scaled up preparation of benzenesulfonic acid salt of sulfasalazine.

FIG. 3 shows a comparison of XRPD diffractograms of the scaled up preparation of benzenesulfonic acid salt of sulfasalazine before and after stability studies, as described in the experimental section below.

FIG. 4 shows a comparison of XRPD diffractograms of the scaled up preparation of diethylamine salt of sulfasalazine before and after stability studies, as described in the experimental section below.

FIG. 5 shows a comparison of XRPD diffractograms of the scaled up preparation of L-lysine salt of sulfasalazine before and after stability studies, as described in the experimental section below.

FIG. 6 shows a comparison of XRPD diffractograms of the scaled up preparation of triethanolamine salt of sulfasalazine before and after stability studies, as described in the experimental section below.

FIG. 7 shows a comparison of XRPD diffractograms of the scaled up preparation of tromethamine salt of sulfasalazine before and after stability studies, as described in the experimental section below.

### DEFINITIONS

The term pKa refers to the negative logarithm (p) of the acid dissociation constant (Ka) of an acid, and is equal to the pH value at which equal concentrations of the acid and its conjugate base form are present in solution.

The term “salt” refers to an ionic compound that results from the neutralization reaction of an acid and a base, and is composed of at least one cation (positively charged ion) and at least one anion (negative ion). In some embodiments, a salt is electrically neutral (without a net charge). In some instances, a salt has a solid form until dissolved in a solvent, e.g., an aqueous solution. An ionic liquid is a salt that has a liquid state. Where applicable, the salt is a pharmaceutically acceptable salt, although this is not required for salts of intermediate compounds that are not intended for administration to a patient. Solid salt forms of sulfasalazine are desirable for use as an Active Pharmaceutical Ingredient (API) in a pharmaceutical composition. By way of example, salts of the present compounds include those wherein the basic compound is protonated by an inorganic or organic acid to form a conjugate acid cation, with the conjugate base of the inorganic or organic acid as the anionic component of the salt. Salts of interest include, but are not limited to, solid crystalline salts. It is understood that for any of the structures depicted herein, such structures may also include any convenient salt forms.

In some embodiments, a “water-soluble” salt is a salt having a solubility in an aqueous solution (e.g., an aqueous buffer at about pH 7 and about 25° C.; or water at about 25° C.) that is 0.1 mg/mL or more, such as a solubility of 0.2 mg/mL or more, 0.3 mg/mL or more, 0.4 mg/mL or more, 0.5 mg/mL or more, 1 mg/mL or more, 2 mg/mL or more, 3 mg/mL or more, 4 mg/mL or more, 5 mg/mL or more, 6 mg/mL or more, 7 mg/mL or more, 8 mg/mL or more, 9 mg/mL or more, 10 mg/mL or more, 15 mg/mL or more, 20 mg/mL or more, or even more.

The term “pharmaceutically acceptable” means approved by a regulatory agency of the Federal or a state government or listed in the U.S. Pharmacopeia or other generally recognized pharmacopeia for use in mammals, such as humans.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, and the like; and when the molecule contains a basic functionality, salts of organic or inorganic acids, such as hydrochloride, and the like.

The term “active pharmaceutical ingredient” (API) refers to a substance or mixture of substances intended to be used in the manufacture of a drug product and that, when used in the production of a drug, becomes an active ingredient in the drug product. Such substances are intended to furnish pharmacological activity or other direct effect in the diagnosis, cure, mitigation, treatment or prevention of disease or to affect the structure and function of the body.

“Dosing interval” in this application means the period of time between administrations of a composition to a patient. For example, if a drug is administered to a patient every 8 hours, then the dosing interval is the 8 hour period that follows the administration of the drug. The condition “for the entire dosing interval” will be considered to be met if the level of the sulfasalazine is at or above the designated level at the end of the dosing interval (but before any next administration of the sulfasalazine).

“Bioavailability” refers the percentage of a dose of a drug that enters the circulation when that dose of the drug is administered orally to a human, rodent or other animal.

“Excipient” is a material used in the compositions of the present application, and may be solid, semisolid or liquid materials which serve as vehicles, carriers or medium for the active compound, such as sulfasalazine. Typical excipients may be found in Remington: The Science and Practice of Pharmacy, A. Gennaro, ed., 20th edition, Lippincott, Williams & Wilkins, Philadelphia, Pa.; Handbook of Pharmaceutical Excipients by Raymond C. Rowe et al. 7th Edition, Pharmaceutical Press, London, UK and The United States Pharmacopeia and National Formulary (USP-NF), Rockville, Md. Excipients may include pharmaceutically acceptable polymers.

“Progressive multiple sclerosis” or “P-MS” refers to all the sub-types of Progressive Multiple Sclerosis characterized by chronic accumulation of disability, which are Primary Progressive Multiple Sclerosis (PP-MS), Secondary Progressive Multiple Sclerosis (SP-MS) and Progressive-Relapsing Multiple Sclerosis (PR-MS).

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric arrangements of the groups described herein are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound. It is understood that the term “or a salt thereof” is intended to include all permutations of salts. It is understood that the term “or a pharmaceutically acceptable salt thereof” is intended to include all permutations of salts. It is understood that the term “or a solvate thereof” is intended to include all permutations of solvates. It is understood that the term “or a stereoisomer thereof” is intended to include all permutations of stereoisomers. It is understood that the term “or a tautomer thereof” is intended to include all permutations of tautomers. Thus for example it follows that it is intended to include a solvate of a pharmaceutically acceptable salt of a tautomer of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

The term “vehicle” refers to a diluent, adjuvant, excipient, or carrier with which a compound of the invention is formulated for administration to a mammal.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a compound” includes a plurality of such compounds and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Before the present compounds and methods are described, it is to be understood that this invention is not limited to particular compounds and methods described, as such may, of course, vary. It is also to be understood that the terminology used herein is for the purpose of describing particular embodiments only, and is not intended to be limiting, since the scope of the present invention will be limited only by the appended claims.

### DETAILED DESCRIPTION

### Sulfasalazine Salt Compositions

As summarized above, the present disclosure relates to various sulfasalazine salts and compositions including the same. Sulfasalazine can be described by the following structure which includes a basic pyridyl group and an acidic salicyclic acid group:

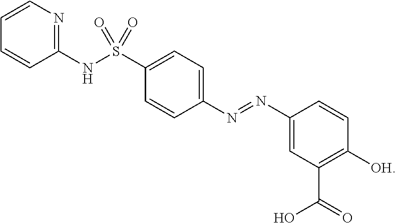 [SMILES: O=C(O)c1cc(/N=N/c2ccc(S(=O)(=O)Nc3ccccn3)cc2)ccc1O]

Sulfasalazine (SSZ) can exist in free acid form (e.g., as depicted above) as a crystalline solid but has poor solubility in most solvents. Since sulfasalazine includes both a basic and an acidic group, the compound can be zwitterionic. Due to the amphoteric nature of sulfasalazine, there is a possibility of sulfasalazine forming salts with acids as well as bases. The present disclosure describes the results of screening for basic salts (i.e., SSZ salt including a counter-anion) and acidic salts (i.e., SSZ salt including a counter-cation) of sulfasalazine which provide one or more desirable properties that are advantageous in the development and preparation of pharmaceutical compositions and methods of using the same.

Aspects of the present disclosure include salts of sulfasalazine. In some instances, the salts of sulfasalazine have a crystalline form. The subject crystalline salts of sulfasalazine can exhibit enhanced water-solubility, e.g., as compared to a zwitterionic or free acid form of sulfasalazine. The term “crystalline” and related terms used herein, when used to describe a substance, component or product, means that the substance, component or product is substantially crystalline as determined by X-ray diffraction, microscopy, polarized microscopy, or other known analytical procedure known to those skilled in the art. See, e.g., Remington's Pharmaceutical Sciences, 18th ed., Mack Publishing, Easton Pa., 173 (1990); The United States Pharmacopeia, 23rd ed., 1843-1844 (1995). In certain instances, the subject crystalline salts of sulfasalazine can exhibit storage stability, e.g., as compared to a zwitterionic or free acid form of sulfasalazine. It is understood that crystalline forms equivalent to the crystalline forms described herein may demonstrate similar, yet non-identical, analytical characteristics within a reasonable range of error, depending on test conditions, purity, equipment and other common variables known to those skilled in the art or reported in the literature.

In some embodiments, the subject salt is a pharmaceutically acceptable basic salt of sulfasalazine and an acid. A basic salt of sulfasalazine is one in which a basic group of sulfasalazine (e.g., the pyridyl N group) is neutralized with the acid to form a salt. Any convenient acids can find use in the preparation of the subject salts (e.g., as described herein). In some cases, the acid used in preparation of the subject salt is an organic sulfonic acid. An organic sulfonic acid is an organosulfur compound of the formula R—S(═O)₂—OH where R is an organic carbon-containing group, such as an alkyl, a substituted alkyl, an aryl, a substituted aryl, a heteroaryl or a substituted heteroaryl. Organic sulfonic acids which find use in preparation of the subject salts include, but are not limited to, benzenesulfonic acid, ethanedisulfonic acid, ethane sulfonic acid, methane sulfonic acid, naphthalene-1,5-disulfonic acid, and p-toluenesulfonic acid. In certain instances, the acid is benzenesulfonic acid. In certain instances, the acid is ethanedisulfonic acid. In certain instances, the acid is ethane sulfonic acid. In certain instances, the acid is methane sulfonic acid. In certain instances, the acid is naphthalene-1,5-disulfonic acid. In certain instances, the acid is p-toluenesulfonic acid. It is understood that a basic salt of sulfasalazine and an organic sulfonic acid can also be referred to as a sulfasalazine sulfonate salt or a sulfasalazine sulfonic acid salt, which terms are used interchangeable herein. As such, in some cases, the salt is sulfasalazine benzenesulfonate. In certain embodiments, the basic salt of sulfasalazine is crystalline.

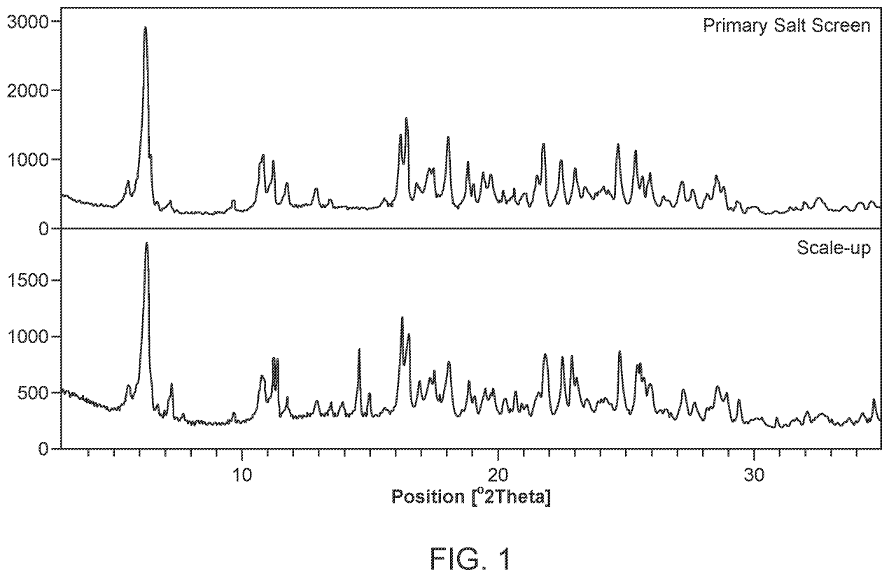

In some embodiments, the crystalline salt is sulfasalazine benzenesulfonic acid (1:1) salt. The crystalline sulfasalazine benzenesulfonic acid salt can have particular polymorph forms that are characterized by an X-ray Powder Diffraction Pattern. In certain cases, the crystalline sulfasalazine benzenesulfonic acid salt is characterized by an X-ray Powder Diffraction Pattern as shown in FIG. 1. In certain cases, the crystalline sulfasalazine benzenesulfonic acid salt is characterized by having a differential scanning calorimetry plot comprising two endothermic events with an onset temperature of about 196° C. and about 204° C. when heated from about 25° C. to about 300° C.

The crystalline forms of the subject salts can be characterized using single crystal data, Powder X-Ray Diffraction (PXRD), Differential Scanning calorimetry (DSC), and/or Thermogravimetric Analysis (TGA). It is to be understood that numerical values described and claimed herein are approximate. Variation within the values may be attributed to equipment calibration, equipment errors, purity of the materials, crystals size, and sample size, among other factors. In addition, variation may be possible while still obtaining the same result. For example, X-ray diffraction values are generally accurate to within +/−0.2 degrees and intensities (including relative intensities) in an X-ray diffraction pattern may fluctuate depending upon measurement conditions employed. Similarly, DSC results are typically accurate to within about 2.degree. C. Consequently, it is to be understood that the crystalline forms of the present disclosure are not limited to the crystalline forms that provide characterization patterns (i.e., one or more of the PXRD, DSC, and TGA) completely identical to the characterization patterns depicted in the accompanying Figures disclosed herein. Any crystalline forms that provide characterization patterns substantially the same as those described in the accompanying Figures fall within the scope of the present disclosure. The ability to ascertain substantially the same characterization patterns is within the purview of one of ordinary skill in the art.

In certain embodiments, the crystalline salt is a pharmaceutically acceptable acid salt of sulfasalazine and an organic amine base, e.g., an organic primary, secondary or tertiary amino base. Any convenient organic amine bases can find use in the subject crystalline salts (e.g., as described herein). Organic amine bases which find use in preparation of the subject crystalline salts include, but are not limited to, diethylamine, L-lysine, triethanolamine, tromethamine, piperazine, benzathine, diethanolamine and L-arginine. In certain embodiments, the base is selected from diethylamine, L-lysine, triethanolamine and tromethamine. In certain embodiments, the base is diethylamine. In certain embodiments, the base is L-lysine. In certain embodiments, the base is triethanolamine. In certain embodiments, the base is tromethamine. In certain embodiments, the base is piperazine. In certain embodiments, the base is benzathine. In certain embodiments, the base is diethanolamine. In certain embodiments, the base is L-arginine.

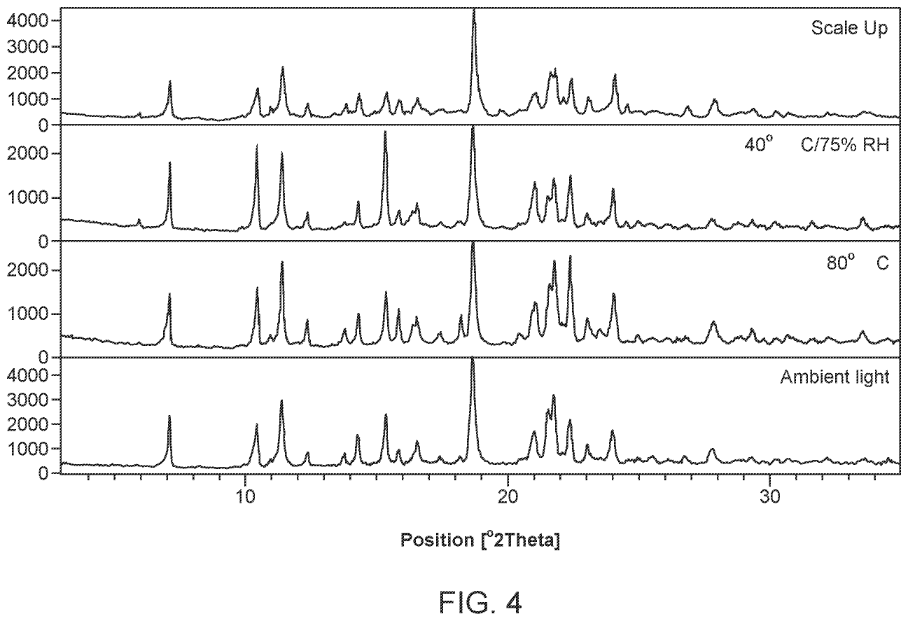

In some embodiments, the crystalline salt is sulfasalazine diethylamine (1:1) salt. In certain instances, the crystalline sulfasalazine diethylamine salt is characterized by the X-ray Powder Diffraction Pattern as shown in FIG. 4. In certain instances, the crystalline sulfasalazine diethylamine salt is characterized by having a differential scanning calorimetry plot comprising one endothermic event with an onset temperature of about 191° C. when heated from about 25° C. to about 300° C.

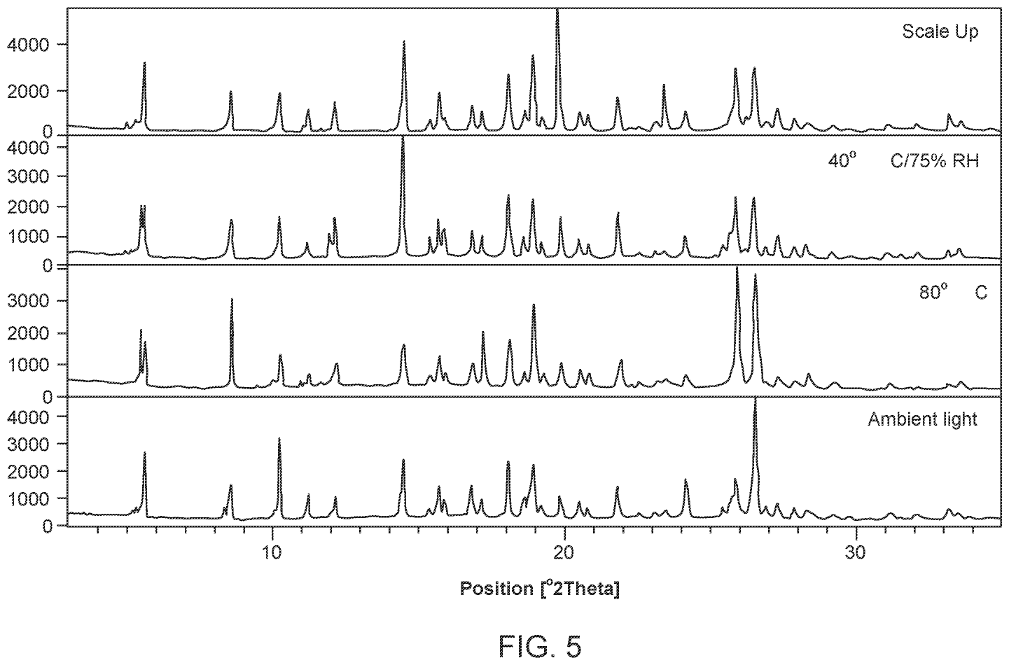

In some embodiments, the crystalline salt is sulfasalazine L-lysine (1:1) salt. In certain instances, the crystalline sulfasalazine L-lysine salt is characterized by the X-ray Powder Diffraction Pattern as shown in FIG. 5. In certain instances, the crystalline sulfasalazine L-lysine salt is characterized by having a differential scanning calorimetry plot comprising no endothermic events when heated from about 25° C. to about 300° C.

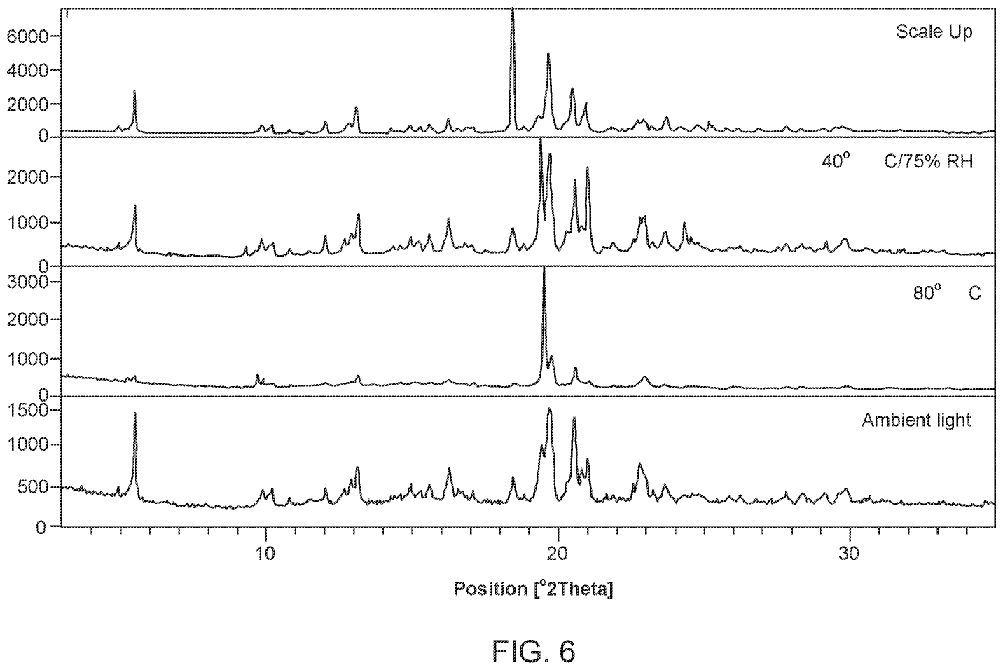

In some embodiments, the crystalline salt is sulfasalazine triethanolamine (1:1) salt. In certain instances, the crystalline sulfasalazine triethanolamine salt is characterized by the X-ray Powder Diffraction Pattern as shown in FIG. 6. In certain instances, the crystalline sulfasalazine triethanolamine salt is characterized by having a differential scanning calorimetry plot comprising one endothermic event with an onset temperature of about 154° C. when heated from about 25° C. to about 300° C.

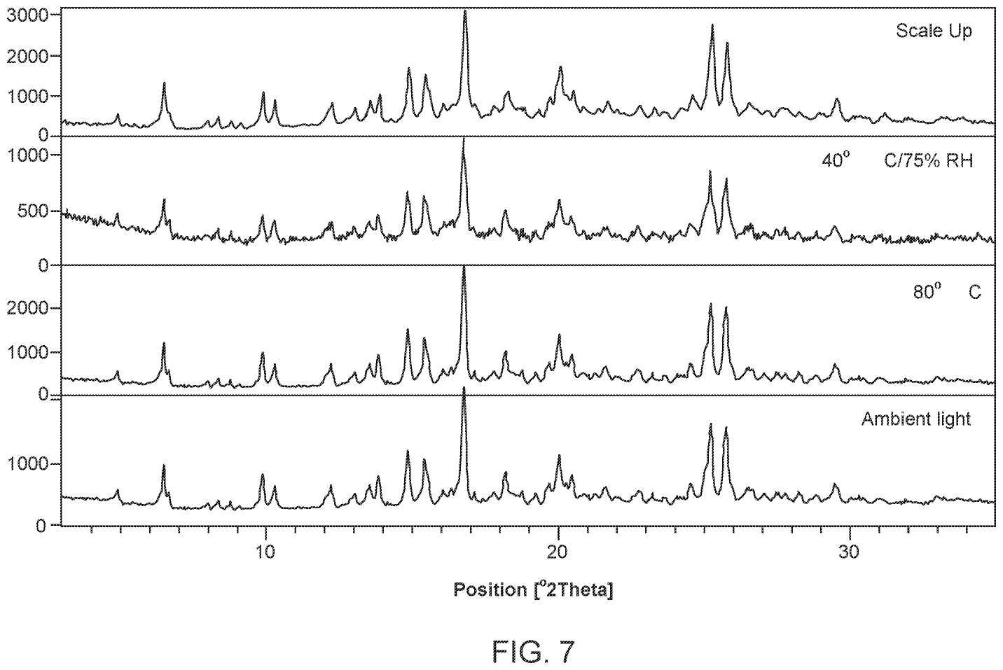

In some embodiments, the crystalline salt is sulfasalazine tromethamine (1:1) salt. In certain instances, the crystalline sulfasalazine tromethamine salt is characterized by the X-ray Powder Diffraction Pattern as shown in FIG. 7. In certain instances, the crystalline sulfasalazine tromethamine salt is characterized by having a differential scanning calorimetry plot comprising endothermic events with an onset temperature of about 67° C. and about 123° C., when heated from about 25° C. to about 300° C.

Aspects of the present disclosure include active pharmaceutical ingredients that include a subject crystalline sulfasalazine salt (e.g., as described herein). An active pharmaceutical ingredient refers to a composition suitable for formulation into a pharmaceutical composition that includes a crystalline sulfasalazine salt (e.g., as described herein), e.g., produced using the subject methods of preparation and optionally be subjected to one or more further purification steps.

In some cases, the subject sulfasalazine salt provides for a substantially non-hygroscopic composition. A non-hygroscopic solid form is desirable for a variety of reasons including, for example, for processing and storage concerns. In some cases, by “substantially non-hygroscopic” is meant a composition that adsorbs 1.0 wt % or less of water, such as 0.9 wt % or less water, 0.8 wt % or less water, 0.7 wt % or less water, 0.6 wt % or less water, 0.5 wt % or less water, 0.4 wt % or less water, 0.3 wt % or less water, 0.2 wt % or less water, or 0.1 wt % or less water, at 90% RH after a Dynamic Vapor Sorption (DVS) cycle, e.g., as described herein. In certain instances, a substantially non-hygroscopic crystalline salt displays a XRPD pattern showing no significant change in crystalline form after a Dynamic Vapor Sorption (DVS) cycle.

The subject sulfasalazine salt can provide for enhanced water-solubility relative to a convenient control form of sulfasalazine, e.g., sulfasalazine in a free acid or zwitterionic form. By “enhanced water-solubility” is meant a form of sulfasalazine that exhibits a solubility in an aqueous solution of interest, by a statistically significant amount, and in some cases by 10% or more, such as 20% or more, 30% or more, 40% or more, 50% or more, 60% or more, 70% or more, 80% or more, 90% or more, 100% or more, 200% or more, 500% or more, 600% or more, 700% or more, 800% or more, 900% or more, 1000% or more, or even more, relative to the solubility of a control form (e.g., free acid form) of sulfasalazine. Any convenient methods can be utilized to assess the solubility of sulfasalazine, including but not limited to, those methods described in the Experimental section below. In some embodiments, the subject crystalline salt has a solubility of 0.1 mg/mL or more in an aqueous buffer at about pH 7 and about 25° C., such as a solubility of 0.2 mg/mL or more, 0.3 mg/mL or more, 0.4 mg/mL or more, 0.5 mg/mL or more, 1 mg/mL or more, 2 mg/mL or more, 3 mg/mL or more, 4 mg/mL or more, 5 mg/mL or more, 6 mg/mL or more, 7 mg/mL or more, 8 mg/mL or more, 9 mg/mL or more, 10 mg/mL or more, 15 mg/mL or more, 20 mg/mL or more, or even more. In some embodiments, the subject crystalline salt has a solubility of 0.1 mg/mL or more in an aqueous solution (e.g., water) at about 25° C., such as a solubility of 0.2 mg/mL or more, 0.3 mg/mL or more, 0.4 mg/mL or more, 0.5 mg/mL or more, 1 mg/mL or more, 2 mg/mL or more, 3 mg/mL or more, 4 mg/mL or more, 5 mg/mL or more, 6 mg/mL or more, 7 mg/mL or more, 8 mg/mL or more, 9 mg/mL or more, 10 mg/mL or more, 15 mg/mL or more, 20 mg/mL or more, or even more.

In some cases, subject sulfasalazine salt provides for a composition that is stable. By “stable” or “stability” is meant a composition that is chemically stable and/or physically stable under conventional temperature and humidity storage conditions (e.g., as described herein, e.g., when maintained at 25° C.) for an extended period of time. By “extended period of time” is meant 1 month or longer, such as 2 months or longer, 3 months or longer, 4 months or longer, including 6 months or longer, e.g., at 1 year or longer, 1.5 years or longer, etc. Chemical stability refers to degradation whereby the chemical nature of the sulfasalazine active agent or salt thereof is changed, e.g., via degradation into less active or inactive structural fragments and derivatives of the compound. The content and impurity levels of chemically stable compositions remained unchanged upon storage. Physical stability refers to degradation of a physical characteristic of the salt, e.g., the crystalline form (e.g., a polymorph) of the salt changes from one form to another form, which in some cases may be less chemically stable or more hygroscopic.

In some cases, the subject crystalline salt is polymorphically stable. In certain instances, the polymorphic stability of the salt is such that at least 90 wt % of the salt, such as at least 91 wt %, at least 92 wt %, at least 93 wt %, at least 94 wt %, at least 95 wt %, at least 96 wt %, at least 97 wt %, at least 98 wt %, at least 99 wt %, maintains its crystal form after exposure to approximately 40° C. and about 75% for 1 week or more, such as 2 weeks or more, 3 weeks or more, 4 weeks or more, 6 weeks or more, 2 months or more, 3 months or more, 4 months or more, 5 months or more, or even 6 months or more.

Aspects of the present disclosure include pharmaceutical compositions including a subject crystalline salt of sulfasalazine (e.g., as described herein) and a pharmaceutically acceptable vehicle. In certain instances, the subject pharmaceutical composition is storage-stable. By “storage-stable” is meant that the salts and compositions may be stored for extended periods of time without significant phase separation and/or significant reduction in activity of the sulfasalazine active agent. In certain embodiments, the subject salts and compositions are stable for 2 months or longer, such as 3 months or longer, 4 months or longer, including 6 months or longer, e.g., at 1 year or longer, 1.5 years or longer, etc., when maintained at 25° C. By the phrase “without substantially decreasing the activity of the sulfasalazine active agent” is meant that at the end of the storage period, there is less than about 10% reduction in activity of the sulfasalazine active agent compared to the beginning of the storage period, such as a 9% or less, 8% or less, 7% or less, 6% or less, 5% or less, 4% or less, 3% or less, 2% or less, or 1% or less reduction in activity. In certain embodiments, the compositions exhibit substantially no change in crystalline form over an extended period of time when maintained at 25° C., where by “extended period of time” is meant 1 month or longer, such as 2 months or longer, 3 months or longer, 4 months or longer, including 6 months or longer, e.g., at 1 year or longer, 1.5 years or longer, etc.

In some instances, a “storage-stable” composition is a composition that maintains a level of sulfasalazine in the composition at 95% or more relative to the level of sulfasalazine that is present before storage, after 1 week or more storage at 40° C./75% relative humidity (RH), such as a level of sulfasalazine of 96% or more, 97% or more, 98% or more, or 99% or more, relative to the level of sulfasalazine that is present before storage. Any convenient methods can be utilized to assess the level of sulfasalazine, including but not limited to HPLC purity analysis methods as described in the Experimental section below.

In some embodiments the subject composition is storage stable for 2 weeks or more after storage at about 40° C. and about 75% relative humidity (RH), such as 3 weeks or more, 4 weeks or more, 6 weeks or more, 2 months or more, 3 months or more, 4 months or more, 5 months or more, or even 6 months or more, e.g., maintains a level of sulfasalazine of 95% or more, 96% or more, 97% or more, 98% or more, or 99% or more, relative to the level of sulfasalazine that is present before storage. In some cases, storage stability refers to chemical stability of sulfasalazine in the composition. In certain cases, storage stability can also refer to physical stability of the crystalline salt form of sulfasalazine, e.g., a crystalline salt form of sulfasalazine that does not revert back to free acid or zwitterionic sulfasalazine.

By “enhanced stability” is meant a composition including a form of sulfasalazine that exhibits an increase in chemical stability of the sulfasalazine active agent, by a statistically significant amount, and in some cases by 10% or more, such as 20% or more, 30% or more, 40% or more, 50% or more, 60% or more, 70% or more, 80% or more, 90% or more, 100% or more, or even more, relative to the half-life of a control form (e.g., free acid form) of sulfasalazine. Any convenient methods can be utilized to assess the degradation of sulfasalazine, including but not limited to, HPLC purity analysis methods as described in the Experimental section below.

By “enhanced water-solubility” is meant a form of sulfasalazine that exhibits a solubility in an aqueous solution of interest, by a statistically significant amount, and in some cases by 10% or more, such as 20% or more, 30% or more, 40% or more, 50% or more, 60% or more, 70% or more, 80% or more, 90% or more, 100% or more, 200% or more, 500% or more, 600% or more, 700% or more, 800% or more, 900% or more, 1000% or more, or even more, relative to the solubility of a control form (e.g., free acid form) of sulfasalazine. Any convenient methods can be utilized to assess the solubility of sulfasalazine, including but not limited to, those methods described in the Experimental section below.

Further aspects of the subject pharmaceutical compositions, and methods of using the same are described in the following sections.

### Methods of Use

Sulfasalazine compositions described herein may be employed in a variety of methods. Aspects of the present disclosure include a method that includes administering to a subject in need thereof a therapeutically effective amount of a sulfasalazine salt or pharmaceutical composition (e.g., as described herein) to treat or prevent a disease or condition of interest. By “therapeutically effective amount” is meant the concentration of a compound that is sufficient to elicit the desired biological effect (e.g., treatment or prevention of epilepsy). Any convenient diseases and indications of interest in which sulfasalazine finds use in treating may be targeted according to the subject methods. Exemplary diseases and conditions of interest which may be targeted for treatment according to the subject methods include, but are not limited to, neurological related diseases (e.g., epilepsy), neurodegenerative diseases, inflammatory conditions and cancers.

The terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance. “Treatment,” as used herein, covers any treatment of a disease in a mammal, in some cases in a human, and includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient. In some instances, the method delays occurrence of a symptom associated with the disease. In certain instances, the method reduces the magnitude of a symptom associated with the disease. In some instances, treating or treatment includes one or more of (1) limiting, inhibiting or reducing the rate of accumulation of disability and/or loss of motor neuron function; (2) delaying the progression of the disease, such as neuropathic pain, neuropathic pain results from painful diabetic neuropathy, or neuropathic pain manifests as dysesthesia, or neuropathic pain manifests as allodynia; rheumatoid arthritis or ankylosing spondylitis; epilepsies and seizure disorders, P-MS or ALS; (3) limiting, inhibiting or reducing neuronal dysfunction and/or muscular atrophy, (4) limiting or arresting its development, (5) relieving the disease, i.e., causing the regression of epilepsies and seizure disorders, P-MS or ALS; (6) reducing or preventing the recurrence of the accumulation of disability and/or the loss of motor neuron function; (7) reducing or preventing the recurrence of neuronal dysfunction and/or muscular atrophy; (8) palliating the symptoms of the disease, (9) increase in survival after onset of epilepsies and seizure disorders, P-MS or ALS; and/or, (10) attenuation of neuroinflammation.

Neurological related diseases of interest which may be targeted for treatment according to the subject methods include, but are not limited to, epilepsy, such as severe subtypes of epilepsy and/or refractory epilepsy, e.g. Dravet syndrome, Lennox-Gastaut syndrome, Doose syndrome, West syndrome, and/or other forms of refractory epilepsy. There are a vast number of subtypes of epilepsy. While all forms of epilepsy are distressing, some subtypes of epilepsy are more severe than others. By “severe” or “refractory,” reference is made to, for example, subtypes of epilepsy that are intractable and/or which are characterized by episodes of status epilepticus. In some embodiments, the subject is diagnosed as having intractable seizures. Intractable seizures are ones that fail to come under control with treatment. These seizures are sometimes also called “uncontrolled” or “refractory.” Subtypes of epilepsy which can be targeted for treatment according to the subject methods include, but are not limited to, Dravet syndrome, Lennox-Gastaut syndrome, Doose syndrome, West syndrome, and/or other types of refractory epilepsy. In certain instances, the subject suffers from a refractory epilepsy, such as Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia, epileptic encephalopathies, and Long-term epilepsy associated tumors (LEATs) for example ganglioglioma, oligodendroglioma, and dysembryoplastic neuroepithelial tumors (DNETs).

In certain embodiments, the subject methods of treatment using a sulfasalazine salt composition can significantly: (1) reduce levels of neuroinflammatory cells in the spinal cord of a subject, including both activated microglia and activated astrocytes, (2) increase the absolute survival and the survival after onset of definitive neurological disease; and/or (3) prevent demyelination in optic neuritis. In certain instances, the method is performed in a mouse model of neurodegeneration.

Neurodegenerative diseases which may be targeted for treatment according to the subject methods include, but are not limited to, Alexander disease, Alzheimer's disease (AD), frontotemporal dementia, HIV-associated dementia, and other dementias, amyotrophic lateral sclerosis, epilepsy, Huntington's disease (HD), ischemic stroke, Motor neurone diseases (MND), neuropathic pain, Parkinson's disease (PD) and PD-related disorders, Prion disease, Rett syndrome, Spinal muscular atrophy (SMA), Spinocerebellar ataxia (SCA), traumatic brain injury, and tuberous sclerosis. In some cases, the neurodegenerative disease or disorder is progressive multiple sclerosis (P-MS), amyotrophic lateral sclerosis (ALS), or is neuropathic pain

Inflammatory diseases and conditions which may be targeted for treatment according to the subject methods include, but are not limited to, inflammatory bowel diseases, such as ulcerative colitis and Crohn's disease, inflammatory arthritis diseases such as ankylosing spondylitis, rheumatoid arthritis and psoriatic arthritis. In certain instance, the subject salt and compositions find use in the treatment of an inflammatory disease or condition such as rheumatoid arthritis.

Cancers of interest which may be targeted for treatment according to the subject methods include, but are not limited to, glial tumors, glioblastoma, lymphoma, pancreatic cancer, etc.

In some embodiments, the composition is administered at a dosage and/or frequency effective to reduce the occurrence of side effects of sulfasalazine. Such side effects can include loss of appetite, nausea, headache, rash, bone marrow suppression, liver problems, and kidney problems.

One challenge with treating various diseases of interest (e.g., as described herein) with pharmaceutical compositions comprising sulfasalazine is the poor oral bioavailability of the conventional formulations of sulfasalazine. For example, only 15% of the sulfasalazine in an orally administered dose of Azulfidine is absorbed into the bloodstream (see Azulfidine Sulfasalazine Tablets Label, LAB-0241-3.0, revised October 2009). In general, because the level of sulfasalazine at the site of action relevant to the disease of interest (e.g., as described herein) is proportional to the amount of sulfasalazine in the plasma, the poor bioavailability of the conventional formulation of sulfasalazine limits the amount of sulfasalazine that reaches some sites of action. For example, in neurodegenerative diseases of interest the site of action can be the spinal cord. Thus, use of a conventional formulation of sulfasalazine to treat a disease of interest (e.g., a neurological or neurodegenerative disease) would require large oral doses of sulfasalazine to be administered. This could expose patients to high levels of sulfasalazine in the gastrointestinal tract and generate high levels of sulfapyridine in the plasma, thereby increasing potential toxicity and side effects. Sulfapyridine was used in the 1940's and 1950's as an antibacterial agent in humans and is a member of the sulfa drugs class. It causes allergic reactions in 3-8% of recipients as reported in medical reviews, which manifest as itching, red rashes, hives or welts, swelling in the throat, vomiting, stomach cramping, diarrhea and in some cases Stevens-Johnson syndrome. Agranulocytosis is a rare but serious side effect of sulfapyridine that increases risk of systemic infections.

The present disclosure addresses these issues, among others, by providing for improved oral bioavailability of sulfasalazine using the subject compositions for the treatment of any of the diseases on interest (e.g., as described herein). Increasing such bioavailability would allow dosing levels of sulfasalazine to be lower, with the further benefit of limiting gastrointestinal exposure to sulfasalazine and systemic exposure to sulfapyridine. In one aspect, there is provided a method for limiting gastrointestinal exposure to sulfasalazine and systemic exposure to sulfapyridine by the administration of a therapeutically effective amount of the pharmaceutical composition as disclosed herein. The formulations disclosed herein can increase the therapeutic index for sulfasalazine in the treatment of any convenient disease. The application provides methods of treating various diseases and disorders using the compositions in which the solubility and/or bioavailability of sulfasalazine has been increased.

Aspects of the subject methods include co-administration of the subject sulfasalazine salt with an ABCG2 inhibitor that can provide for desirable in vivo pharmacokinetic properties of the sulfasalazine active agent. Co-administration is meant to include simultaneous or sequential administration of the subject sulfasalazine salt with an ABCG2 inhibitor. As such, the ABCG2 inhibitor can be administered as part of the same composition as the sulfasalazine salt or administered separately. In certain embodiments, there are provided methods for treating a disease or disorder (e.g., as described herein) in a patient comprising orally administering to the patient one or more pharmaceutical composition(s) comprising a therapeutically effective amount of sulfasalazine salt (e.g., as described herein) and, either separately or together, an ABCG2 inhibitor. In some embodiments, the method comprises orally administering to the patient in need thereof a pharmaceutical composition comprising a therapeutically effective amount of sulfasalazine salt, an ABCG2 inhibitor, optionally a polymer, and a pharmaceutically acceptable excipient, where the disease or disorder is a neurological related disease, a neurodegenerative disease, an inflammatory condition and cancer (e.g., as described herein).

The term ABCG2 inhibitor is an acronym for ATP-binding cassette sub-family G member 2. ATP-binding cassette sub-family G member 2 is a protein that in humans is encoded by the ABCG2 gene, see Allikmets R, et al. Hum Mol Genet. 5: 1649-55 (1997) and Doyle L. et al. Proc Natl Acad Sci U.S.A. 95: 15665-70 (1999). ABCG2 has also been designated as CDw338 (cluster of differentiation w338). The membrane-associated protein encoded by this gene is included in the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). ABCG2 protein is a member of the White subfamily. Alternatively referred to as the Breast Cancer Resistance Protein, this protein functions as a xenobiotic transporter which may play a role in multi-drug resistance to chemotherapeutic agents including mitoxantrone and camptothecin analogues.

Examples of ABCG2 inhibitors which may find use in the subject methods and compositions include, but are not limited to, N-[4-[2-(3,4-Dihydro-6,7-dimethoxy-2(1H)-isoquinolinyl)ethyl]phenyl]-9,10-dihydro-5-methoxy-9-oxo-4-acridinecarboxamide (elecridar); 2-chloro-N-(4-chloro-3-(pyridin-2-yl)phenyl)-4-(methylsulfonyl)benzamide (HhAntag691); (3S,6S,12aS)-1,2,3,4,6,7,12,12a-Octahydro-9-methoxy-6-(2-methylpropyl)-1,4-dioxopyrazino[1′,2′:1,6]pyrido[3,4-b]indole-3-propanoic acid 1,1-dimethylethyl ester(raltegravir); N-(4-Methyl-3-((4-(pyridin-3-yl)pyrimidin-2-yl)amino)phenyl)-4-((4-methylpiperazin-1-yl)methyl)benzamide (imatinib); Fumitremorgin C; 4-[4-[[4-chloro-3-(trifluoromethyl)phenyl]carbamoylamino]phenoxy]-N-methylpyridine-2-carboxamide; 4-methylbenzenesulfonic acid (sorafenib); (1E,6E)-1,7-bis (4-hydroxy-3-methoxyphenyl)-1,6-heptadiene-3,5-dione (curcumin) and Cathomycin sodium. In some cases, the subject pharmaceutical composition can include a polymer. The polymer used can be biocompatible, pharmaceutically acceptable and water soluble. The polymer may be a copolymer of vinylpyrrolidone with vinyl acetate and as such can be any PVP VA polymer that is water soluble including PVP VA64.

In some cases, the ABCG2 inhibitor is selected from the group consisting of tocopheryl polyethyleneglycol succinate (TPGS), polysorbate (Tween) and Pluronic. In certain aspects, the ABCG2 inhibitor is TPGS. In some instances, the ABCG2 inhibitor is a non-ionic compound. In certain cases, the ABCG2 inhibitor is a GRAS compound. In some instances, the ABCG2 is selected from the group consisting of TPGS, Tocophersolan (e.g., TPGS), and polysorbate, polysorabate-20 (Tween-20), Brij30, Cremphor EL, and Pluronic compounds, Pluronic P85 and Pluronic L21. In some aspects, the pharmaceutical formulation is a solid dose formulation, wherein the formulation comprises a polymer selected from PVP VA64 or HPMCAS. In another aspect, the pharmaceutical formulation is a liquid formulation that does not comprise a polymer such as PVP VA64 or HPMCAS. In some aspects, the formulation comprises between 1 mg and 500 mg of the ABCG2 inhibitor, such as TPGS per dose, such as 10 mg, 100 mg, 200 mg, 300 mg, 400 mg or 500 mg. In some aspects, the ratio of the sulfasalazine to PVP VA64 or HPMCAS in the pharmaceutical composition is about 20:80 wt/wt to 50:50 wt/wt, or about 25:75 wt/wt. In another aspect, the in vitro solubility of the sulfasalazine salt is at least 500 μg/ml. In yet another aspect, the in vitro solubility of the sulfasalazine salt is between about 500 μg/ml and 11,500 μg/ml.

In certain embodiments, the subject pharmaceutical compositions comprise sulfasalazine salt (e.g., as described herein) and an inhibitor of the ABCG2 efflux transporter (i.e., ABCG2 efflux inhibitors or ABCG2 inhibitors). In some cases, the compositions can be used to treat neurodegenerative diseases and disorders. In one aspect, the ABCG2 efflux inhibitor is selected from the group consisting of Pluronic P85, Tween 20, E-TPGS (TPGS), Pluronic 85, Brij 30, Pluronic L81, Tween 80 and PEO-PPO, or mixtures thereof. In another aspect, the ABCG2 inhibitor is TPGS or Tween 20, or a mixture thereof. In another aspect, the ABCG2 inhibitor is TPGS. In one variation, the composition comprises one ABCG2 inhibitor, or a mixture of two or more ABCG2 inhibitors.

The subject compositions and methods can provide for desirable in vivo pharmacokinetic properties and parameters of the sulfasalazine active. Depending on the application, pharmaceutical formulations with improved or equivalent Cmax, Tmax, T_{1/2}, and/or bioavailability can be utilized in the subject methods. The pharmacokinetic profiles of the subject formulation can have pharmacokinetic profiles where one or more pharmacokinetic parameters are improved compared to the pharmacokinetic parameters seen with an identical formulation made with an equal molar quantity of a zwitterionic or free base form of sulfasalazine. Useful pharmacokinetic parameters in which to compare formulations include maximal blood therapeutic concentration (Cmax), time to reach Cmax (Tmax), time to reach a blood concentration of ½ of Cmax (T_{1/2}) and bioavailability (BA). BA can be measured by determining an area under the curve (AUC) of a blood therapeutic concentration versus time graph. For comparative analysis between pharmaceutical compositions, the pharmacokinetic parameters can be compared individually, or in various combinations.

In certain embodiments, the presence of an ABCG2 inhibitor increases the oral bioavailability of sulfasalazine salt by at least 25%, at least 50%, at least 100%, at least 150%, at least 200%, at least 250%, or at least 300% higher than the plasma level of sulfasalazine after administration of the same dose level of a control sample of sulfasalazine, as measured in the blood plasma. In one embodiment, the compositions comprising sulfasalazine and the ABCG2 inhibitor are a solid oral dose. In other embodiments, the sulfasalazine and the ABCG2 inhibitor comprises a liquid suspension or solution. In certain embodiments, the ABCG2 inhibitor comprises 0.01% to 90%, such as 0.01% or more by weight, such as 0.05% or more, 0.1% or more, 0.5% or more, 1% or more, 5% or more, 10% or more, 20% or more, 30% or more, 40% or more or 50% or more by weight of the total pharmaceutical composition. In certain embodiments, the ABCG2 inhibitor comprises 0.01% to 200% by weight relative to sulfasalazine salt, such as 0.01%, 0.05%, 0.1%, 0.5%, 1%, 5%, 10%, 20%, 30%, 40% and 50% by weight relative to sulfasalazine salt (i.e., ABCG2 inhibitor:sulfasalazine salt) in the therapeutic composition.

Neurological related diseases of interest which may be targeted for treatment according to the subject methods involving co-administration of the subject sulfasalazine salt with an ABCG2 inhibitor include, but are not limited to, epilepsy, such as severe subtypes of epilepsy and/or refractory epilepsy, e.g. Dravet syndrome, Lennox-Gastaut syndrome, Doose syndrome, West syndrome, and/or other forms of refractory epilepsy, such as Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia and epileptic encephalopathies.

Neurodegenerative diseases which may be targeted for treatment according to the subject methods involving co-administration of the subject sulfasalazine salt with an ABCG2 inhibitor include, but are not limited to, Alexander disease, Alzheimer's disease (AD), frontotemporal dementia, HIV-associated dementia, and other dementias, amyotrophic lateral sclerosis, epilepsy, Huntington's disease (HD), ischemic stroke, Motor neurone diseases (MND), neuropathic pain, Parkinson's disease (PD) and PD-related disorders, Prion disease, Rett syndrome, Spinal muscular atrophy (SMA), Spinocerebellar ataxia (SCA), traumatic brain injury, tuberous sclerosis, progressive multiple sclerosis (P-MS), amyotrophic lateral sclerosis (ALS), and neuropathic pain.

Inflammatory diseases and conditions which may be targeted for treatment according to the subject methods involving co-administration of the subject sulfasalazine salt with an ABCG2 inhibitor include, but are not limited to, inflammatory bowel diseases, such as ulcerative colitis and Crohn's disease, inflammatory arthritis diseases such as ankylosing spondylitis, rheumatoid arthritis and psoriatic arthritis.

Cancers of interest which may be targeted for treatment according to the subject methods involving co-administration of the subject sulfasalazine salt with an ABCG2 inhibitor include, but are not limited to, glial tumors, glioblastoma, lymphoma, pancreatic cancer, etc.

In some embodiments of the subject method involving co-administration of the subject sulfasalazine salt with an ABCG2 inhibitor, the seizure disease or disorder is selected from the group consisting of Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia, epileptic encephalopathies, and Long-term epilepsy associated tumors (LEATs) for example ganglioglioma, oligodendroglioma, and dysembryoplastic neuroepithelial tumors (DNETs).

In some aspects of the subject methods involving co-administration of the subject sulfasalazine salt with an ABCG2 inhibitor, the neurodegenerative disease is selected from progressive multiple sclerosis and other demyelinating diseases, including, but not limited to, Acute Disseminated Encephalomyelitis, Adrenoleukodystrophy, Adrenomyeloneuropathy, Chronic Axonal Neuropathy, Chronic Inflammatory Demyelinating Polyneuropathy or CIDP, Chronic Relapsing Polyneuropathy, Devic Disease, Guillian-Barre Syndrome, HIV induced CIDP, Leber's Hereditary Optic Neuropathy, Lewis Sumner variant of CIDP, Multifocal Acquired Demyelinating Sensory and Motor Neuropathy, Multifocal Motor Neuropathy, NeuromyelitisOptica, Optic Neuritis, Paraproteinaemic Demyelinating Neuropathy, Tropical Spastic Paraparesis, amyotrophic lateral sclerosis, Alzheimer's disease, Parkinson's disease, epilepsy and other seizure disorders, including but not limited to Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glutl Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, epileptic encephalopathies, Focal Cortical Dysplasia, and Tuberous Sclerosis Complex, neuropathic pain, Huntington's disease, ischemic stroke, traumatic brain injury, concussion, Rett Syndrome, Frontotemporal Dementia, HIV-associated Dementia and Alexander disease.

In certain embodiments, the present application discloses pharmaceutical compositions comprising sulfasalazine in a formulation suitable for intravenous (IV) dosing. In one aspect, the IV formulation contains an ABCG2 inhibitor. These formulations are suitable for acute care treatment, especially for treatment of ischemic stroke, traumatic brain injury, seizure disorders and demyelinating diseases.

In some embodiments, a single dose of the subject compound is administered. In other embodiments, multiple doses of the subject compound are administered. Where multiple doses are administered over a period of time, the subject compound is administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

In certain embodiments, methods are provided for treating a patient comprising orally administering to the patient a pharmaceutical composition comprising sulfasalazine salt, wherein the dose of the pharmaceutical composition is sufficient to maintain a plasma level of sulfasalazine of at least 8 μg/ml for at least 14 total hours a day. In certain embodiments, a plasma level of sulfasalazine of at least 8 μg/ml is maintained for between 21 and 24, inclusive, total hours a day. In certain embodiments, a plasma level of sulfasalazine of at least 8 μg/ml is maintained for 24 hours a day. In certain embodiments, the dose of the pharmaceutical composition is sufficient to maintain a plasma level of sulfasalazine of between about 8 μg/ml and 30 μg/ml, inclusive, or between about 8 μg/ml and 16 μg/ml, inclusive, or between about 10 μg/ml and 16 μg/ml, inclusive, for the given amount of time; or for the entire dosing interval. For the purposes of this application, the condition “for the entire dosing interval” will be considered to be met if the level of the sulfasalazine is at or above the designated level at the end of the dosing interval (but before any next administration of the sulfasalazine). In certain embodiments, the dose of the pharmaceutical composition is sufficient to produce a plasma level of sulfasalazine in the patient of between about 8 μg/ml and 30 μg/ml, between about 10 μg/ml and 30 μg/ml, between about 8 μg/ml and 16 μg/ml or between about 8 μg/ml and 12 μg/ml, inclusive; at least 10 μg/ml, or 16 μg/ml for the entire dosing interval.

One way to increase plasma levels of sulfasalazine is to administer higher daily doses of a conventional formulation of sulfasalazine to patients. In humans, plasma levels of sulfasalazine are proportional to the oral dose, e.g. Khan et al, Gut 21:232-240 (1980). In certain embodiments, the present disclosure provides methods for treating a patient comprising orally administering to the patient a pharmaceutical composition comprising sulfasalazine, an ABCG2 inhibitor and a pharmaceutically acceptable excipient, wherein the total daily dose of sulfasalazine salt is between about 2.5 grams and 8 grams, or between about 3 grams and 5 grams, inclusive; or about 3 grams, about 4 grams, or about 5 grams.

In certain embodiments, the subject is human. As used herein, the terms “host”, “subject”, “individual” and “patient” are used interchangeably and refer to any mammal in need of such treatment according to the disclosed methods. Exemplary mammals include, but are not limited to, humans, domestic animals (e.g., a dog, cat, or the like), farm animals (e.g., a cow, a sheep, a pig, a horse, or the like) or laboratory animals (e.g., a monkey, a rat, a mouse, a rabbit, a guinea pig, or the like). In certain embodiments, the subject is human. “Patient” refers to human and non-human subjects, especially mammalian subjects.

Administration of the subject pharmaceutical compositions may be systemic or local. In certain embodiments, administration to a mammal will result in systemic release of sulfasalazine (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compositions of the present disclosure are administered orally. In certain embodiments, it may be desirable to administer one or more compounds of the invention locally to the area in need of treatment. This may be achieved, for example, by local infusion during, topical application, by injection, by means of a catheter, by means of a suppository, or by means of an implant, said implant being of a porous, non-porous, or gelatinous material, including membranes, such as sialastic membranes, or fibers.

The dose of sulfasalazine administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

The amount of compound administered can be determined using any convenient methods to be an amount sufficient to produce the desired effect in association with a pharmaceutically acceptable diluent, carrier or vehicle. The specifications for the unit dosage forms of the present disclosure will depend on the particular compound employed and the effect to be achieved, and the pharmacodynamics associated with each compound in the host. The dose administered to an animal, particularly a human, in the context of the present disclosure should be sufficient to effect a prophylactic or therapeutic response in the animal over a reasonable time frame, e.g., as described in greater detail herein. Dosage levels of the order of from about 0.01 mg to about 140 mg/kg of body weight per day are useful in representative embodiments, or alternatively about 0.5 mg to about 7 g per patient per day. Dosage will depend on a variety of factors including the particular salt employed, the condition of the animal, and the body weight of the animal, as well as the severity of the illness and the stage of the disease. The size of the dose will also be determined by the existence, nature, and extent of any adverse side-effects that might accompany the administration of a particular salt.

In certain embodiments, the compound is administered as a pharmaceutical preparation. In embodiments of the invention, any effective dose of the subject sulfasalazine salt or composition can be employed. The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, such as 25 mg, 50 mg, 100 mg, 200 mg, 300 mg, 400 mg, 500 mg, 600 mg, 800 mg, or 1000 mg.

In some embodiments, there is provided a pharmaceutical composition (e.g., as described herein) wherein the pharmaceutical composition has been formulated such that the in vitro solubility of the sulfasalazine salt is between about 500 μg/ml and about 11,500 μg/ml; or is between about 500 μg/ml and about 7,500 μg/ml, about 500 μg/ml and about 5,500 μg/ml, about 500 μg/ml and about 2500 μg/ml, between about 2300 μg/ml and 11,500 μg/ml; or at least 500 μg/ml, 1200 μg/ml or 2300 μg/ml. In one aspect, the solubility is determined at a pH of 5.5 using any convenient method. In some aspects, the “in vitro solubility” of sulfasalazine is considered to be the Cmax IB at 90 minutes. In certain instances, the pharmaceutical composition comprises sulfasalazine, an ABCG2 inhibitor and a pharmaceutically acceptable excipient.

Combination therapy includes administration of a single pharmaceutical dosage formulation which contains the subject composition and one or more additional agents; as well as administration of the subject composition and one or more additional agent(s) in its own separate pharmaceutical dosage formulation. For example, a subject composition and an additional agent active with antiepileptic activity can be administered to the patient together in a single dosage composition such as a combined formulation, or each agent can be administered in a separate dosage formulation. Where separate dosage formulations are used, the subject composition and one or more additional agents can be administered concurrently, or at separately staggered times, e.g., sequentially. Antiepileptic agent of interest that find use in combination therapies of the present disclosure include, but are not limited to, Acetazolamide, Carbamazepine, Clobazam, Clonazepam, Eslicarbazepine acetate, Ethosuximide, Gabapentin, Lacosamide, Lamotrigine, Levetiracetam, Nitrazepam, Oxcarbazepine, Perampanel, Piracetam, Phenobarbital, Phenytoin, Pregabalin, Primidone, Retigabine, Rufinamide, Sodium valproate, Stiripentol, Tiagabine, Topiramate, Vigabatrin and Zonisamide.

In certain aspects of the combination therapies, a patient with P-MS is administered (or co-administered with) Mitoxantrone, Gilenya, Masitinib, Siponimod, Tcelna, Tecfidera, Lemtrada, Laquinimod, Daclizumab, Ocrelizumab, Cladribine, Daclizumab, Tysabri, Campath, Rituximab, Fingolimod, Azathioprine or Ibudilast.

In some embodiments, methods for treating a patient with a neurodegenerative disease or disorder comprise administering to the patient an effective amount of an inhibitor of system x_{c}⁻ other than sulfasalazine are provided. In certain embodiments, the system x_{c}⁻ inhibitor is selected from (S)-4-carboxyphenylglycine, 2-hydroxy-5-((4-(N-pyridin-2-ylsulfamoyl)phenyl)ethynyl)benzoic acid, aminoadipate (AAA),4-(1-(2-(3,5-bis(trifluoromethyl)phenyl)hydrazono)ethyl)-5-(4 (trifluoromethyl)benzyl)isoxazole-3-carboxylic acid, 5-benzyl-4-(1-(2-(3,5-bis(trifluoro-methyl)phenyl)hydrazono)ethyl)isoxazole-3-carboxylic acid and 2-hydroxy-5-[2-[4-[(3-methylpyridin-2-yl)sulfamoyl]phenyl]ethynyl] benzoic acid.

In some embodiments, the subject method is an in vitro method that includes contacting a sample with a subject composition. The protocols that may be employed in these methods are numerous, and include but are not limited to, cell-free assays, binding assays (e.g., receptor binding assays); cellular assays in which a cellular phenotype is measured, e.g., gene expression assays; and assays that involve a particular animal model for a condition of interest (e.g., Tuberous Sclerosis Complex).

### Pharmaceutical Compositions

Also provided are pharmaceutical compositions or preparations that include sulfasalazine active pharmaceutical ingredient compositions, e.g., prepared according to the subject methods. Pharmaceutical compositions can include a crystalline sulfasalazine salt composition (either alone or in the presence of one or more additional active agents) present in a pharmaceutically acceptable vehicle. In some embodiments, the pharmaceutical composition includes crystalline sulfasalazine salt composition (e.g., as described herein) as the only active agent formulated in a pharmaceutically acceptable excipient.

The choice of excipient will be determined in part by the particular salt, as well as by the particular method used to administer the composition. Accordingly, there is a wide variety of suitable formulations of the subject pharmaceutical composition.

The dosage form of sulfasalazine employed in the methods of the present disclosure can be prepared by combining the crystalline sulfasalazine salt composition with one or more pharmaceutically acceptable diluents, carriers, adjuvants, and the like in a manner known to those skilled in the art of pharmaceutical formulation.

As described above, the subject compositions can include an absorption enhancer and/or an efflux inhibitor. In some cases, the subject composition includes an inhibitor of the ABCG2 efflux transporter (i.e., ABCG2 efflux inhibitors or ABCG2 inhibitors), e.g., in an amount effective to provide a desirable bioavailability of sulfasalazine in a subject (e.g., as described herein). Examples of ABCG2 inhibitors that can be included in the subject compositions are described herein.

As described above, the subject compositions can include a polymer, e.g., a biocompatible and pharmaceutically acceptable polymer. In some cases, the polymer is water soluble. The polymer may be a copolymer of vinylpyrrolidone with vinyl acetate and as such can be any PVP VA polymer that is water soluble including PVP VA64. In certain embodiments, the pharmaceutically acceptable polymer may be selected from polyvinylpyrrolidone (PVP, including PVP VA64, homo- and copolymers of polyvinylpyrrolidone and homopolymers or copolymers of N-vinylpyrrolidone); crospovidone; polyoxyethylene-polyoxypropylene copolymers (also known as poloxamers); cellulose derivatives (including hydroxypropyl methyl cellulose acetate succinate (HPMCAS), hydroxypropyl methyl cellulose phthalate (HPMCP), hydroxypropyl methylcellulose (HPMC), cellulose acetate phthalate (CAP), cellulose acetate trimellitate (CAT), hydroxypropyl methyl cellulose acetate phthalate, hydroxypropyl methyl cellulose acetate trimellitate, cellulose acetate succinate, methylcellulose acetate succinate, carboxymethyl ethyl cellulose (CMEC), hydroxypropyl methyl cellulose, hydroxypropyl methyl cellulose acetate, hydroxyethylcellulose); dextran; cyclodextrins; homo- and copolymers of vinyllactam, and mixtures thereof; gelatins; hypromellose phthalate; sugars; polyhydric alcohols; polyethylene glycol (PEG); polyethylene oxides; polyoxyethylene derivatives; polyvinyl alcohol; propylene glycol derivatives and the like; SLS; Tween; EUDRAGIT (a methacrylic acid and methyl methacrylate copolymer); and combinations thereof. The polymer may be water soluble or water insoluble. In certain embodiments, the ratio of the sulfasalazine to polymer in the composition is about 5:95 wt/wt to 50:50 wt/wt. In certain embodiments, the wt/wt ratio of the ABCG2 inhibitor to sulfasalazine (ABCG2:sulfasalazine) in the composition may be about 1:1. 1:2. 1:3, 1:4, 1:5, 1:10, 1:20, 1:50, 1:100 or about 1:200; or may be about 1:20 wt/wt.

The subject compositions can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient (sulfasalazine), as solids, pellets or granules; (c) suspensions in an appropriate liquid; and (d) suitable emulsions Tablets and capsules of interest include ones that provide for immediate release of active agent from the formulation, and tablets and capsules that provide for controlled release, e.g., over an extended period of time (e.g., as described herein). Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein. Solid forms such as pellets or granules can be coated or uncoated. Solid forms such as pellets or granules can in some cases provide for immediate release of active agent from the formulation, and in other cases provide for controlled release, e.g., over an extended period of time (e.g., as described herein).

The subject pharmaceutical composition can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers.

By way of illustration, the sulfasalazine pharmaceutical composition can be admixed with conventional pharmaceutically acceptable carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1% to about 90% by weight of the active compound, and more generally from about 1% to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

Particular formulations of the present disclosure are in a liquid form. The liquid may be a solution or suspension and may be an oral solution or syrup which is included in a bottle with a pipette which is graduated in terms of milligram amounts which will be obtained in a given volume of solution. The liquid solution makes it possible to adjust the solution for small children which can be administered anywhere from 0.5 mg to 15 mg and any amount between in half milligram increments and thus administered in 0.5, 1.0, 1.5, 2.0 mg, etc.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable or dispersible powder or granules.

### Methods of Preparation

Also provided are methods of preparing the subject crystalline sulfasalazine salts. In some embodiments, the method comprising: a) combining sulfasalazine and an organic sulfonic acid (e.g., as described herein) or an organic amine base (e.g., as described herein) in an organic solvent (e.g., as described herein) under conditions sufficient to crystallize a particular sulfasalazine salt (e.g., as described herein); and b) isolating the sulfasalazine salt.

In certain instances, step a) includes neutralizing sulfasalazine with an organic sulfonic acid. In some cases, the organic sulphonic acid is selected from benzenesulfonic acid, ethanedisulfonic acid, ethane sulfonic acid, methane sulfonic acid, naphthalene-1,5-disulfonic acid and p-toluenesulfonic acid. In certain instances of the method, the solvent is selected from acetone, acetonitrile, dioxance, ethanol, isopropyl alcohol (IPA) and tetrahydrofuran (THF). In certain embodiments of the method, the acid is benzenesulfonic acid and the solvent is acetonitrile.

In certain instances, step a) includes neutralizing sulfasalazine with an organic amine base. In some cases, the organic amine base is selected from diethylamine, L-lysine, triethanolamine, tromethamine, piperazine, benzathine, diethanolamine and L-arginine. In certain embodiments of the method, the solvent is selected from acetone, acetonitrile, dioxance, ethanol, isopropyl alcohol (IPA) and tetrahydrofuran (THF). In certain embodiments of the method, the organic amine base is diethylamine and the solvent is ethanol. In certain embodiments of the method, the organic amine base is L-lysine and the solvent is acetone. In certain embodiments of the method, the organic amine base is triethanolamine and the solvent is acetone. In certain embodiments of the method, the organic amine base is tromethamine and the solvent is ethanol.

In some case, the method further comprises drying the sulfasalazine salt. In certain instances, the method further comprises formulating the sulfasalazine salt with a pharmaceutically acceptable excipient to obtain a pharmaceutical composition.

### Additional Embodiments

Additional embodiments are set forth in the following clauses.

Clause 1. A water-soluble crystalline salt of sulfasalazine.

Clause 2. The crystalline salt of clause 1, wherein the crystalline salt is substantially non-hygroscopic.

Clause 3. The crystalline salt of clause 1, wherein the crystalline salt has a solubility of 1 mg/mL or more in an aqueous buffer at about pH 7 and 25° C.

Clause 4. The crystalline salt of clause 1, wherein the crystalline salt is polymorphically stable.

Clause 5. The crystalline salt of clause 1, wherein the sulfasalazine of the crystalline salt is storage stable.

Clause 6. The crystalline salt of clause 4 or 5, wherein at least 90 wt % of the crystalline salt maintains its crystal form after exposure to approximately 40° C. and approximately 75% for approximately 1 week.

Clause 7. The crystalline salt of any one of clauses 1-6, wherein the crystalline salt is a pharmaceutically acceptable basic salt of sulfasalazine and an acid.

Clause 8. The crystalline salt of clause 7, wherein the acid is an organic sulfonic acid.

Clause 9. The crystalline salt of clause 7 or 8, wherein the acid is selected from benzenesulfonic acid, ethanedisulfonic acid, ethane sulfonic acid, methane sulfonic acid, naphthalene-1,5-disulfonic acid, p-toluenesulfonic acid and sulfuric acid.

Clause 10. The crystalline salt of clause 9, wherein the acid is benzenesulfonic acid.

Clause 11. The crystalline salt of any one of clauses 1-6, wherein the crystalline salt is a pharmaceutically acceptable acid salt of sulfasalazine and an organic amine base.

Clause 12. The crystalline salt of clause 11, wherein the base is selected from diethylamine, L-lysine, triethanolamine, tromethamine, piperazine, benzathine, diethanolamine and L-arginine.

Clause 13. The crystalline salt of clause 12, wherein the base is selected from diethylamine, L-lysine, triethanolamine and tromethamine.

Clause 14. A crystalline sulfasalazine benzenesulfonic acid (1:1) salt.

Clause 15. The crystalline salt of clause 14, characterized by a X-ray Powder Diffraction Pattern as shown in FIG. 1.

Clause 16. The crystalline salt of clause 14, characterized by having a differential scanning calorimetry plot comprising two endothermic events with an onset temperature of about 196° C. and about 204° C. when heated from about 25° C. to about 300° C.

Clause 17. A crystalline sulfasalazine diethylamine (1:1) salt.

Clause 18. The crystalline salt of clause 17, characterized by the X-ray Powder Diffraction Pattern as shown in FIG. 4.

Clause 19. The crystalline salt of clause 17, characterized by having a differential scanning calorimetry plot comprising one endothermic event with an onset temperature of about 191° C. when heated from about 25° C. to about 300° C.

Clause 20. A crystalline sulfasalazine L-lysine (1:1) salt.

Clause 21. The crystalline salt of clause 20, characterized by the X-ray Powder Diffraction Pattern as shown in FIG. 5.

Clause 22. The crystalline salt of clause 20, characterized by having a differential scanning calorimetry plot comprising no endothermic events when heated from about 25° C. to about 300° C.

Clause 23. A crystalline sulfasalazine triethanolamine (1:1) salt.

Clause 24. The crystalline salt of clause 23, characterized by the X-ray Powder Diffraction Pattern as shown in FIG. 6.

Clause 25. The crystalline salt of clause 23, characterized by having a differential scanning calorimetry plot comprising one endothermic event with an onset temperature of about 154° C. when heated from about 25° C. to about 300° C.

Clause 26. A crystalline sulfasalazine tromethamine (1:1) salt.

Clause 27. The crystalline salt of clause 26, characterized by the X-ray Powder Diffraction Pattern as shown in FIG. 7.

Clause 28. The crystalline salt of clause 26, characterized by having a differential scanning calorimetry plot comprising endothermic events with an onset temperature of about 67° C. and about 123° C., when heated from about 25° C. to about 300° C.

Clause 29. A pharmaceutical composition comprising the crystalline salt of any one of clauses 1-28 and a pharmaceutical acceptable carrier, diluent or excipient.

Clause 30. The pharmaceutical composition of clause 29, wherein the composition is storage stable.

Clause 31. The composition of clause 30, wherein the crystalline salt is storage stable for one week or more at 40° C. and 75% RH.

Clause 32. The composition of clause 31, wherein the crystalline salt comprises about 95% or more by weight of the crystalline salt after storage at 40° C. and 75% relative humidity for 1 week.

Clause 33. The composition of clause 29, wherein the composition is formulated for oral administration.

Clause 34. The composition of clause 29, wherein the composition is formulated for parenteral administration.

Clause 35. The composition of clause 29, wherein the composition is formulated for intravenous administration.

Clause 36. The composition of clause 29, wherein the composition is formulated as a single unit dosage form.

Clause 37. The composition of clause 29, wherein the dosage form is a tablet or capsule.

Clause 38. The composition of clause 29, wherein the dosage form is a pellet or granule.

Clause 39. A method of treating disease or condition that is a neurological related disease, a neurodegenerative disease, an inflammatory disease or condition or cancer, the method comprising administering to a subject in need thereof a therapeutically effective amount of a crystalline salt of any one of clauses 1-28, or a pharmaceutical composition according to any one of clauses 29-37.

Clause 40. The method of clause 39, wherein the disease or condition is a neurological related disease.

Clause 41. The method of clause 40, wherein the neurological related disease is epilepsy.

Clause 42. The method of clause 41, wherein the epilepsy is refractory epilepsy.

Clause 43. The method of clause 42, wherein the subject is diagnosed as having intractable seizures.

Clause 44. The method of clause 41 or 42, wherein the epilepsy is selected from Dravet syndrome, Lennox-Gastaut syndrome, Doose syndrome, West syndrome, Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glut 1 Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia, epileptic encephalopathies, and Long-term epilepsy associated tumors (LEATs) for example ganglioglioma, oligodendroglioma, and dysembryoplastic neuroepithelial tumors (DNETs).

Clause 45. The method of clause 39, wherein the disease or condition is a neurodegenerative disease.

Clause 46. The method of clause 45, wherein the neurodegenerative disease is selected from Alexander disease, Alzheimer's disease (AD), frontotemporal dementia, HIV-associated dementia and other dementias, amyotrophic lateral sclerosis, epilepsy, Huntington's disease (HD), ischemic stroke, Motor neurone diseases (MND), neuropathic pain, Parkinson's disease (PD) and PD-related disorders, Prion disease, Rett syndrome, Spinal muscular atrophy (SMA), Spinocerebellar ataxia (SCA), traumatic brain injury, tuberous sclerosis, progressive multiple sclerosis (P-MS), amyotrophic lateral sclerosis (ALS) and neuropathic pain.

Clause 47. The method of clause 39, wherein the disease or condition is an inflammatory disease or condition.

Clause 48. The method of clause 47, wherein the disease or condition is an inflammatory disease or condition is selected from inflammatory bowel diseases, ulcerative colitis, Crohn's disease, inflammatory arthritis diseases, ankylosing spondylitis, rheumatoid arthritis and psoriatic arthritis.

Clause 49. The method of clause 39, wherein the disease or condition is cancer.

Clause 50. The method of clause 49, wherein the cancer is selected from glial tumors, glioblastoma, lymphoma and pancreatic cancer.

Clause 51. The method of clause 40, wherein the composition is administered at a dosage and/or frequency effective to reduce the occurrence of side effects of sulfasalazine.

Clause 52. The method of clause 41, further comprising co-administering to the subject an antiepileptic agent.

Clause 53. The method of any one of clauses 39-51, further comprising co-administering to the subject an ABCG2 inhibitor.

Clause 54. The method of clause 53, wherein the ABCG2 inhibitor and the crystalline salt of sulfasalazine are co-formulated in a single pharmaceutical composition.

Clause 55. A method of preparing a crystalline sulfasalazine salt, the method comprising:

a) combining sulfasalazine and an organic sulphonic acid in an organic solvent under conditions sufficient to crystallize a sulfasalazine salt; and

b) isolating the sulfasalazine salt;

wherein the organic sulphonic acid is selected from benzenesulfonic acid, ethanedisulfonic acid, ethane sulfonic acid, methane sulfonic acid, naphthalene-1,5-disulfonic acid and p-toluenesulfonic acid.

Clause 56. The method of clause 55, wherein the solvent is selected from acetone, acetonitrile, dioxance, ethanol, isopropyl alcohol (IPA) and tetrahydrofuran (THF).

Clause 57. The method of clause 55, wherein the acid is benzenesulfonic acid and the solvent is acetonitrile.

Clause 58. The method of any one of clauses 55-57, further comprising drying the sulfasalazine salt.

Clause 59. The method of any one of clauses 55-57, further comprising formulating the sulfasalazine salt with a pharmaceutically acceptable excipient to obtain a pharmaceutical composition.

Clause 60. A method of preparing a crystalline sulfasalazine salt, the method comprising:

a) combining sulfasalazine and an organic amine base in an organic solvent under conditions sufficient to crystallize a sulfasalazine salt; and

b) isolating the sulfasalazine salt;

wherein the organic amine base is selected from diethylamine, L-lysine, triethanolamine, tromethamine, piperazine, benzathine, diethanolamine and L-arginine.

Clause 61. The method of clause 60, wherein the solvent is selected from acetone, acetonitrile, dioxance, ethanol, isopropyl alcohol (IPA) and tetrahydrofuran (THF).

Clause 62. The method of clause 61, wherein:

a) the organic amine base is diethylamine and the solvent is ethanol

b) the organic amine base is L-lysine and the solvent is acetone;

c) the organic amine base is triethanolamine and the solvent is acetone; or

d) the organic amine base is tromethamine and the solvent is ethanol.

Clause 63. The method of any one of clauses 60-62, further comprising drying the sulfasalazine salt.

Clause 64. The method of any one of clauses 60-63, further comprising formulating the sulfasalazine salt with a pharmaceutically acceptable excipient to obtain a pharmaceutical composition.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. By “average” is meant the arithmetic mean. Standard abbreviations may be used, e.g., s or sec, second(s); min, minute(s); h or hr, hour(s); and the like.

### General Methods of Analysis

### X-Ray Powder Diffraction (XRPD)

X-ray powder diffraction (XRD) is a rapid analytical technique used for phase identification of a crystalline material and can provide information on unit cell dimensions. XRPD analysis is carried out on a PANalytical X'pert pro, scanning the samples between 3 and 35° 2θ. The material is gently ground to release any agglomerates and loaded onto a multi-well plate with Mylar polymer film to support the sample. The multi-well plate is then placed into the diffractometer and analyzed using Cu K radiation (α1λ=1.54060 Å; α2=1.54443 Å; β=1.39225 Å; α1:α2 ratio=0.5) running in transmission mode (step size 0.0130° 20) using 40 kV/40 mA generator settings.

### Polarised Light Microscopy (PLM)

The presence of crystallinity (birefringence) is determined using an Olympus BX50 polarizing microscope, equipped with a Motic camera and image capture software (Motic Images Plus 2.0). All images are recorded using the 20× objective, unless otherwise stated.

### Thermogravimetric Analysis (TGA)

Approximately, 5 mg of material is weighed into an open aluminium pan and loaded into a simultaneous thermogravimetric/differential thermal analyser (TG/DTA) and held at room temperature. The sample is then heated at a rate of 10° C.·min⁻¹ from 20° C. to 350° C. during which time the change in sample weight was recorded along with any differential thermal events (DTA). Nitrogen is used as the purge gas at a flow rate of 300 cm³·min⁻¹.

### Differential Scanning Calorimetry (DSC)

Approximately 5 mg of material is weighed into an aluminum DSC pan and sealed non-hermetically with a pierced aluminum lid. The sample pan is then loaded into a Seiko DSC6200 (equipped with a cooler) cooled and held at 20° C. Once a stable heat-flow response is obtained, the sample and reference are heated to 220° C. at a scan rate of 10° C.·min-1 and the resulting heat flow response monitored. Nitrogen is used as the purge gas at a flow rate of 50 cm³·min⁻¹.

### Dynamic Vapor Sorption (DVS)

Approximately, 10-20 mg of sample was placed into a mesh vapor sorption balance pan and loaded into a DVS Intrinsic dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Determination of Solubility at Enteric pH

The release of sulfasalazine from a pharmaceutical composition can be determined using the following procedure. A sample mass of 4.5 mg of the test material is placed in a microcentrifuge tube. To this is added 0.9 mL of gastric buffer (GB) solution (0.01 N HCl, pH 2). The tubes are vortexed for one minute, then centrifuged for one minute before taking each sample. Samples (the liquid phase) are taken at 5, 15, and 25 minutes. At 30 minutes after the start of the test, 0.9 mL of intestinal buffer (IB) solution (a phosphate/citrate buffer at pH 5.5) is added to the tubes (at a double concentration of the buffer salts to result in the desired pH level and buffer strength). The tubes are vortexed for one minute, then centrifuged for one minute before taking each sample. Samples are taken at 4, 10, 20, 40, 90 and 1200 minutes after addition of the intestinal buffer solution. The concentration of sulfasalazine is determined by HPLC.

### Example 1

### Screening Acids for Formation of Basic Salts of Sulfasalazine

Salt screening was performed to identify crystalline and developable salt forms of sulfasalazine. The screening produced a number of pharmaceutically acceptable salts of sulfasalazine which have desirable physical properties for further development, in some cases with significantly higher aqueous solubility compared to the free base. This work focuses on using the pyridine functionality of sulfasalazine which has a measured pKa of ˜8.05. Due to the wide variety of acidic salt formers available with this basic pKa, an extensive salt screen of 24 acids in six solvent systems was performed.

### Solvent Solubility

100 μL aliquots of solvent were added to approximately 10 mg sulfasalazine. Between each addition, the mixture was checked for dissolution and where no dissolution was apparent, the mixture was heated to ca. 40° C. and checked again. This procedure was continued until dissolution was observed or until 2 mL of solvent had been added. Table 1 below contains a list of solvents used in the solvent solubility screen.

TABLE 1

| | Solvent | ICH Class |
|---|---|---|
| 1 | Acetone | 3 |
| 2 | Acetone/water (50:50) | 3 |
| 3 | Acetonitrile | 2 |
| 4 | Acetonitrile/water (50:50) | 2 |
| 5 | 1-Butanol | 3 |
| 6 | Dichloromethane | 2 |
| 7 | Dimethylformamide | 2 |
| 8 | 1,4-Dixoane | 2 |
| 9 | Ethanol | 3 |
| 10 | Ethyl acetate | 3 |
| 11 | Ethyl formate | 3 |
| 12 | Hepatane | 3 |
| 13 | Isobutyl acetate | 3 |
| 14 | Isopropyl acetate | 3 |
| 15 | Methanol | 2 |
| 16 | Methanol/water (50:50) | 2 |
| 17 | Methylethyl Ketone | 3 |
| 18 | Nitromethane | 2 |
| 19 | 1-Propanol | 3 |
| 20 | 2-Propanol | 3 |
| 21 | 2-Propanol/water (50:50) | 3 |
| 22 | Tetrahydrofuran | 2 |
| 23 | Toluene | 2 |
| 24 | Water | n/a |

### Primary Salt Screen

The primary salt screen was carried out using stock solutions of 24 acids (1M), which were prepared in various solvents. These were added to the free acid sulfasalazine suspended in a selection of six solvents. The procedure is described below. The acids and solvents used in the study can be found in Table 2 and Table 3 respectively.
Approximately 50 mg of sulfasalazine was weighed into each of 144 vials;
1 mL of the appropriate solvent was added to form a suspension of the solid;
131.8 μL of the appropriate acid stock solution (1.05 equivalents) was added to each vial;
The resulting samples were then temperature cycled between ambient and 40° C. in 4 hour cycles for ca. 72 hours. Where dissolution had occurred, the solvent was allowed to evaporate so that solids could be recovered;
Any solids produced were analyzed by XRPD, TG/DTA and 1H NMR where material amounts allowed. Furthermore, stability studies at 40° C. and 75% RH were carried out on solids for ca. 48 hours.

TABLE 2
List of Primary Salt Screen Acids

| | Acid |
|---|---|
| 1 | Acetic acid |
| 2 | Adipic Acid |
| 3 | Benzenesulfonic acid |
| 4 | Benzoic acid |
| 5 | Citric acid |
| 6 | Ethanedisulfonic acid |
| 7 | Ethanesulfonic acid |
| 8 | Fumaric acid |
| 9 | Glycolic acid |
| 10 | Hippuric acid |
| 11 | Hydrochloric acid |
| 12 | L-Ascorbic acid |
| 13 | L-Lactic acid |
| 14 | L-Malic acid |
| 15 | L-Tartaric acid |
| 16 | Malonic acid |
| 17 | Methanesulfonic acid |
| 18 | Naphthalene-1,5-disulfonic acid |
| 19 | Pamoic acid |
| 20 | Phosphoric acid |
| 21 | p-Toluenesulfonic acid |
| 22 | Succinic acid |
| 23 | Sulfuric acid |
| 24 | Thiocyanic acid |

TABLE 3
List of Primary Salt Screen Solvents

| | Solvent | ICH Class |
|---|---|---|
| 1 | Acetone | 3 |
| 2 | Acetonitrile | 2 |
| 3 | Dioxane | 2 |
| 4 | Ethanol | 3 |
| 5 | IPA | 3 |
| 6 | THF | 2 |

### Secondary Salt Screen and Developability Assessment

After the primary salt screen, one salt of interest was scaled up. The salt was produced on a 500 mg scale using the following procedure:
Approximately 500 mg of sulfasalazine was accurately weighed into a 20 mL scintillation vial;
10 mL of the appropriate solvent was added to the solid to create a suspension;
A stock solution of the acid was prepared to 1M concentration in water;
A volume of the appropriate stock solution (1318 μL, 1.05 equivalents) was added to the suspension;
The vial was then shaken for ca. 72 hours while temperature cycling between ambient and 40° C. in 4 hour cycles;
The solid was filtered off and dried under vacuum at ambient temperature for ca. 3 hours;
A yield of each experiment was calculated and the solids were analyzed using the following techniques: XRPD; TG/DTA; DSC; DVS (with post-DVS XRPD analysis); IR; 1H NMR; UPLC; 1-week stability assessments at: 40° C./75% RH, 80° C.; or under ambient light; Salt disproportionation studies with XRPD analysis and pH measurement; Hydration studies with post XRPD analysis; Thermodynamic solubility studies with post XRPD analysis.

The procedures for the last four techniques are detailed in the sections below.

### Stability Assessments

Approximately 20 mg of the salt was weighed into a glass vial. A separate sample was prepared for each set of conditions:
40° C./70% RH—the sample was placed in an oven at 40° C. Inside the oven the sample was placed in a dessicator containing saturated sodium chloride solution to maintain the humidity 75% RH;
80° C.—the sample was placed in an oven at 80° C.;
Ambient light—the sample was placed on a bright windowsill at ambient temperature.
   After 1 week, XRPD and HPLC purity analysis was performed on the solids.

### Salt Disproportionation Studies

Approximately 50 mg of the salt was weighed into a glass vial. 1.0 mL of deionised water was added and the sample was slurried at ambient temperature for ca. 24 hrs. Pre- and post-agitation pH readings were taken and the sample analyzed by XRPD after agitation.

### Hydration Studies

Approximately 50 mg of each salt was weighed into a glass vial. Three different mixtures of H₂O and acetone, which corresponded to three different water activites, were prepared to a total volume of 10 mL. The preparation is detailed in Table 4. A volume of the corresponding solvent mixture was added until a slurry was formed, and then the samples were agitated at ambient temperature for ca. 12 hrs. Pre- and post-agitation pH readings were performed, as well as post-agitation XRPD analysis on any remaining solid.

TABLE 4
Solvent Stock Solution Preparations for the Hydration Studies

| Approximate | Volume | Volume | Stock Volume |
|---|---|---|---|
| Water Activity | H₂O (μl) | Acetone (μl) | Added (μl) |
| 0.281 | 60 | 9940 | 1000 |
| 0.572 | 260 | 9740 | 1000 |
| 0.790 | 2680 | 7320 | 1000 |

### Thermodynamic Solubility Studies

Buffers at pH values of 1, 4.5 and 6.8 were prepared. Firstly, 0.2 M components for the buffers were made in water (Table 5). Then the buffers were prepared by combining the different components and adjusting the pH to the required value (Table 6). Approximately 20 mg of the salt was weighed into a glass vial, to which was added 0.5 mL of the corresponding buffer solution to create a slurry. The samples were then agitated at ambient temperature for ca. 24 hrs. Pre- and post-agitation pH readings were taken and any remaining solid analyzed by XRPD. Furthermore, the concentration of the solutions was determined by HPLC analysis.

TABLE 5
Preparation of Buffer Components

| | Mass/Volume | Volume Made |
|---|---|---|
| Component | (g/mL) | Up To With H₂O |
| Hydrochloric Acid (37%) | 1.8 | 100 |
| Potassium Chloride | 1.49 | 100 |
| Potassium Hydrogen | 4.09 | 100 |
| Phthalate | | |
| Potassium Phosphate | 2.72 | 100 |
| Monobasic | | |
| Sodium Hydroxide | 0.8 | 100 |

TABLE 6
Preparation of pH Buffers

| | Buffer pH | | |
|---|---|---|---|
| | 1.0 | 4.5 | 6.8 |
| Aqueous | Hydrochloric acid | Sodium hydroxide | Potassium phosphate |
| Component 1 | solution (0.2M) | solution (0.2M) | monobasic solution (0.2M) |
| Volume (mL) | 67 | 4.35 | 25 |
| Aqueous | Potassium chloride | Potassium hydrogen | Sodium hydroxide |
| Component 2 | solution (0.2M) | phthalate solution (0.2M) | solution (0.2M) |
| Volume (mL) | 25 | 25 | 11.2 |
| Volume made up | 100 | 100 | 100 |
| To with H₂O (mL) | | | |

### Results

Solvent Solubility Screen

The solvent solubility screen was performed in 24 solvent systems. The solubility of sulfasalazine was generally very low with solubility values of <5 mg·mL⁻¹ in all solvents except DMF and THF (Table 7). XRPD analysis of the remaining solids found:
Solid with XRPD patterns consistent with the input material was returned from all solvents except 1,4-dioxane, DMF and THF;
No solid was returned from DMF;
A pattern different to the input was returned from 1,4-dioxane and THF.

TABLE 7
Results from the Solubility Screen

| | | Approx. Solubility |
|---|---|---|
| | Solvent | (mg · mL⁻¹) |
| 1 | Acetone | <5 |
| 2 | Acetone/water (50:50) | <5 |
| 3 | Acetonitrile | <5 |
| 4 | Acetonitrile/water (50:50) | <5 |
| 5 | 1-Butanol | <5 |
| 6 | Dichloromethane | <5 |
| 7 | Dimethylformamide | >100 |
| 8 | 1,4-Dixoane | <5 |
| 9 | Ethanol | <5 |
| 19 | Ethyl acetate | <5 |
| 11 | Ethyl formate | <5 |
| 12 | Hepatane | <5 |
| 13 | Isobutyl acetate | <5 |
| 14 | Isopropyl acetate | <5 |
| 15 | Methanol | <5 |
| 16 | Methanol/water (50:50) | <5 |
| 17 | Methylethyl Ketone | <5 |
| 16 | Nitromethane | <5 |
| 19 | 1-Propanol | <5 |
| 20 | 2-Propanol | <5 |
| 21 | 2-Propanol/water (50:50) | <5 |
| 22 | Tetrahydrofuran | 10 |
| 23 | Toluene | <5 |
| 24 | Water | <5 |

### Primary Salt Screen

The primary salt screen was carried out with 24 acids in 6 solvents. Solids with a unique XRPD pattern were further analyzed by TG/DTA and ¹H NMR. Where two solids had the same pattern, the most crystalline solid by XRPD was analyzed.

### 1. Acetic Acid

The primary salt screen with acetic acid after temperature cycling returned:
Crystalline material from all solvent systems (FIG. 5);
Patterns were consistent with the input material for Acetone, Acetonitrile, Ethanol and IPA;
A different pattern from 1,4-dioxane and THF.
   The solid from 1,4-Dioxane was analyzed by TG/DTA and ¹H NMR.

### 2. Adipic Acid

The primary salt screen with adipic acid returned:
Crystalline material from all solvent systems (FIG. 8);
Patterns were consistent with the input material for Acetone, Acetonitrile, Ethanol and IPA;
A different pattern from 1,4-Dioxane and THF. These are the same as the patterns from 1,4-dioxane and THF from acetic acid—these solvates are seen throughout the screen.

### 3. Benzenesulfonic Acid

The primary salt screen with benzenesulfonic acid returned:
Crystalline material from all solvents except acetone and THF
Patterns were consistent with the input material for Ethanol and IPA;
Partially crystalline material from acetone;
Insufficient material from THF—solid was obtained after evaporation (FIG. 14);
A different pattern from input material with acetonitrile and 1,4-dioxane.
   Two of the solids were analyzed by TG/DTA and/or ¹H NMR.

### 4. Benzoic Acid

The primary salt screen with benzoic acid returned:
Crystalline material from all solvent systems;
Patterns were consistent with the input material for Acetone, Acetonitrile, Ethanol and IPA and THF;
A different pattern from 1,4-dioxane—a dioxane solvate.

### 5. Citric Acid

The primary salt screen with citric acid returned:
Crystalline material from all solvent systems;
Patterns were consistent with the input material for Acetone, Acetonitrile, Ethanol and IPA;
A different pattern from 1,4-dioxane and THF—solvates.

### 6. Ethanedisulfonic Acid

The primary salt screen with ethanedisulfonic acid returned:
Crystalline material from all solvent systems;
Different XRPD patterns from all solvents.
   All solids were analyzed by TG/DTA and ¹H NMR.

### 7. Ethanesulfonic Acid

The primary salt screen with ethanesulfonic acid returned:
Crystalline material from all solvent systems;
Three different XRPD patterns, one from acetone, 2-propanol and THF, a second from 1,4-dioxane and the third from acetonitrile and ethanol.
   All solids were analyzed by TG/DTA and ¹H NMR.

### 8. Fumaric Acid

The primary salt screen with fumaric acid returned:
Crystalline material from all solvent systems;
Patterns consistent with the input material from Acetone, Acetonitrile, Ethanol, IPA and THF;
A different pattern from 1,4-dioxane—solvate.

### 9. Glycolic Acid

The primary salt screen with glycolic acid returned:
Crystalline material consistent with the input material from all solvents.

### 10. Hippuric Acid

The primary salt screen with hippuric acid returned:
Crystalline material from all solvent systems;
Patterns were consistent with the input material for Acetone, Acetonitrile, Ethanol, IPA and THF;
A different pattern from 1,4-dioxane—solvate.

### 11. Hydrochloric Acid

The primary salt screen with hydrochloric acid returned:
Crystalline material from all solvent systems;
Patterns were consistent with the input material for Acetonitrile, Ethanol and IPA;
A different pattern from Acetone, 1,4-dioxane and THF.
   The solid from THF was analyzed by TG/DTA and ¹H NMR.

### 12. L-Ascorbic Acid

The primary salt screen with L-Ascorbic acid returned:
Crystalline material from all solvent systems except THF, for which there was insufficient material—crystalline material was returned after evaporation and was consistent with the input material;
Patterns from all solvents except 1,4-dioxane that were consistent with the input material;
The solid from 1,4-dioxane was the 1,4-dioxane solvate.

### 13. L-Lactic Acid

The primary salt screen with L-Lactic acid returned:
Crystalline material consistent with the input material from all solvents.

### 14. L-Malic Acid

The primary salt screen with L-Malic acid returned:
Crystalline material from all solvent systems except 1,4-Dioxane;
Mostly amorphous material from 1,4-dioxane;
Patterns consistent with the input from all solvents except 1,4-dioxane and THF, for which a pattern consistent with their respective solvates was observed.

### 15. L-Tartaric Acid

The primary salt screen with L-Tartaric acid returned:
Crystalline material from all solvent systems;
Patterns were consistent with the input material for Acetone, Acetonitrile, Ethanol and IPA;
A different pattern from 1,4-dioxane and THF—solvates.
   The solid from THF was analyzed by TG/DTA.

### 16. Malonic Acid

The primary salt screen with malonic acid returned:
Crystalline material from all solvent except 1,4-dioxane;
Mostly amorphous material from 1,4-dioxane;
Patterns consistent with the input for all solvents except 1,4-dioxane, for which additional peaks were observed.

### 17. Methanesulfonic Acid

The primary salt screen with methanesulfonic acid returned:
Crystalline material from all solvent systems;
Two different patterns, one from acetone, acetonitrile, ethanol and 2-propanol and one from 1,4-dioxane and THF.
   One solid of each pattern was analyzed by TG/DTA and ¹H NMR.

### 18. Naphthalene-1,5-Disulfonic Acid

The primary salt screen with naphthalene-1,5-disulfonic acid returned:
Crystalline material from all solvent systems;
Six different patterns, all of which were different from the input material.
   The solids from acetone, acetonitrile, 2-propanol and THF were analyzed by TG/DTA.

### 19. Pamoic Acid

The primary salt screen with pamoic acid returned:
Crystalline material from all solvent system);
Patterns consistent with a mixture of the input material and free pamoic acid from all solvent systems.

### 20. Phosphoric Acid

The primary salt screen with phosphoric acid returned:
Crystalline material from all solvent systems;
Patterns consistent with the input material from Acetone, Acetonitrile, Ethanol and IPA;
A different pattern from 1,4-dioxane and THF—solvates.
   21. p-Toluenesulfonic Acid

The primary salt screen with p-toluenesulfonic acid returned:
Crystalline material from all solvents except THF, for which there was insufficient material;
Patterns were consistent with the input material for Acetone, Acetonitrile, Ethanol and IPA;
A different pattern from 1,4-dioxane.
Insufficient material from THF—solid was obtained after evaporation;
The solid from THF after evaporation gave a different XRPD pattern from the solid from 1,4-dioxane after temperature cycling.
   The solids from 1,4-dioxane and THF were analyzed by TG/DTA.

### 22. Succinic Acid

The primary salt screen with succinic acid returned:
Crystalline material from all solvent systems;
Patterns were consistent with the input material for Acetone, Acetonitrile, Ethanol, IPA and THF;
A different pattern from 1,4-dioxane—solvate formation.

### 23. Sulfuric Acid

The primary salt screen with sulfuric acid returned:
Crystalline material from all solvent systems;
Patterns were consistent with the input material for Ethanol and IPA;
A different pattern from Acetone, Acetonitrile, 1,4-dioxane and THF.
   The solids from acetone and THF were analyzed by TG/DTA.

### Secondary Salt Screen and Full Developability Assessment

After the primary salt screen, the benezenesulfonic acid salt was progressed to the secondary screen using acetonitrile as the solvent. The results from the scale up of the benzenesulfonic acid salt are detailed below:

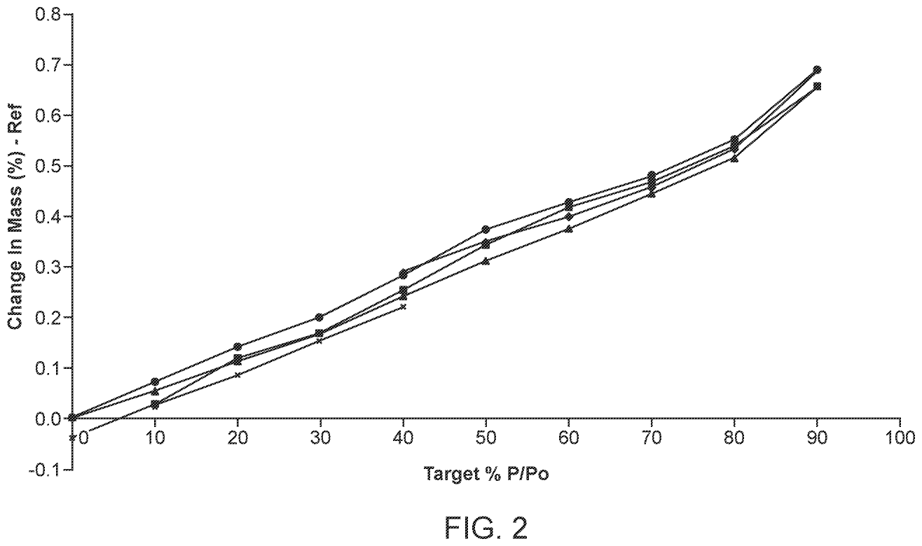

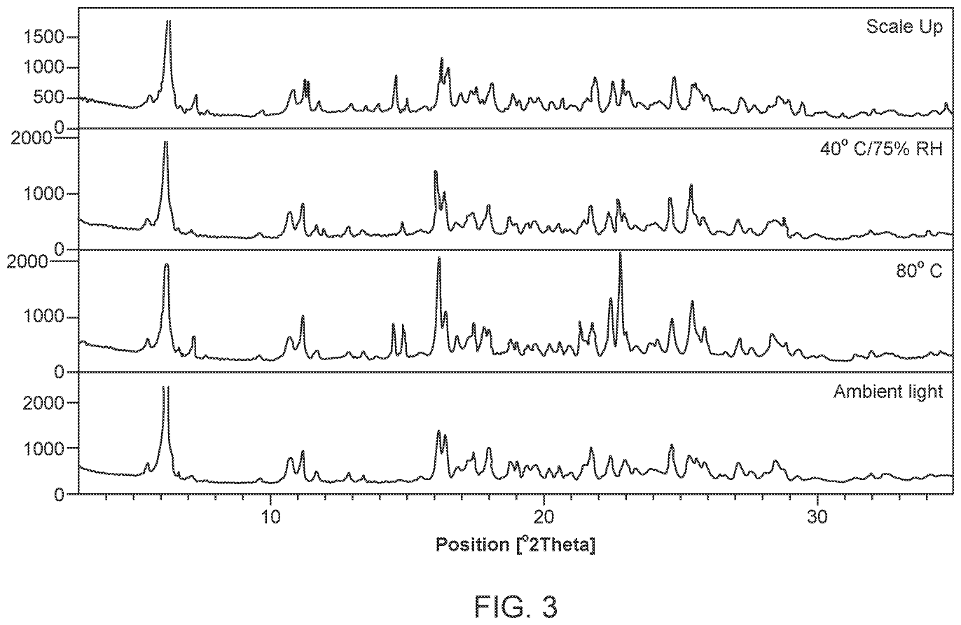

Benzenesulfonic Acid from Acetonitrile
XRPD analysis found the scaled up solid to have the same XRPD pattern as the solid from the primary screen (FIG. 1);
By TGA the solid had a mass loss of ca. 0.3% up to decomposition. A melting event was observed in the DTA with two peaks with onset temperatures at ca. 193 and 204° C. before exothermic decomposition at 264° C.;
There were two endothermic events in the DSC for the first heating cycle, with onset temperature ca. 196 and 204° C., agreeing with the DTA. There were no events in the cooling or second heating cycles;
The DVS isotherm plot (FIG. 2) showed the solid to be minimally hygroscopic, with a mass uptake of ca. 0.7% at 90% RH. In the kinetic DVS plot, no obvious change in solid form occurred. After DVS analysis, the XRPD pattern of the solid remained unchanged;
An IR spectrum of the scaled up solid was taken for reference. A broad peak corresponding to the presence of an —OH group could be seen at ca. 2800 cm-1;
¹H NMR analysis showed the salt to be a 1:1 ratio of acid to base. Only trace amounts of the solvent, acetonitrile, were present;
The salt had a purity of 98.1% by HPLC;
After 1-week stability studies on the solid the following results were found (FIG. 3):

TABLE 8
Purity Values of the Scaled Up Benzenesulfonic
Acid Salt After Stability Studies

| | Average |
|---|---|
| Condition | Purity |
| 40° C./75% RH | 97.3 |
| 80° C. | 98.1 |
| Ambient light | 98.1 |

### Summary of Results

### Primary Salt Screen

The tables below (Table 9 and Table 10) summarize the results of the primary acid salt screen on sulfasalazine:

TABLE 9
Summary of Results of the Primary Salt
Screen for the First Twelve Acids.

| | Ace- | Aceto- | 1,4- | Etha- | 2- | |
|---|---|---|---|---|---|---|
| | tone | nitrile | Dioxane | nol | Propanol | THF |
| Acetic acid | In | In | Di | In | In | In/Th |
| Adipic acid | In | In | Di | In | In | In/Th |
| Benzenesulfonic | In | 1 ¥ | 1 ‡ | In | In | 1 ‡ * |
| acid | | | | | | |
| Benzoic acid | In | In | Di | In | In | In |
| Citric acid | In | In | Di | In | In | In/Th |
| Ethanedisulfonic | 1 ‡ | 2 ‡ | 3 ‡ | 4 ‡ | 5 ‡ | 6 ‡ |
| acid | | | | | | |
| Ethanesulfonic acid | 1 ‡ | 2 ‡ | 3 ‡ | 2 ‡ | 1 ‡ | 1 ‡ |
| Fumaric acid | In | In | Di | In | In | In |
| Glycolic acid | In | In | In | In | In | In |
| Hippuric acid | In | In | Di | In | In | In |
| Hydrochloric acid | 1 ‡ | In | 1 ‡ | In | In | 1 ‡ |
| L-Ascorbic acid | In | In | Di | In | In | In * |

¥ Scaled Up
‡ Crystalline
In Input material
Di Dioxane solvate
Th THF solvate
An asterisk (*) indicates that the solid was obtained from evaporation.

TABLE 10
Summary of Results of the Primary Salt
Screen for the Second Twelve Acids.

| | Ace- | Aceto- | 1,4- | Etha- | 2- | |
|---|---|---|---|---|---|---|
| | tone | nitrile | Dioxane | nol | Propanol | THF |
| L-Lactic acid | In | In | In | In | In | In |
| L-Malic acid | In | In | In/Di | In | In | In/Th |
| L-Tartaric acid | In | In | Di | In | In | Th |
| Malonic acid | In | In | 1 Ω | In | In | In |
| Methanesulfonic | 1 ‡ | 1 ‡ | 2 ‡ | 1 ‡ | 1 ‡ | 2 ‡ |
| acid | | | | | | |
| Naphthalene-1,5- | 1 ‡ | 2 ‡ | 3 ‡ | 4 ‡ | 5 ‡ | 6 ‡ |
| disulfonic acid | | | | | | |
| Pamoic acid | In/Ac | In/Ac | In/Ac | In/Ac | In/Ac | In/Ac |
| Phosphoric acid | In | In | In/Di | In | In | In/Th |
| p-Toluenesulfonic | In | In | 1 ‡ | In | In | 2 * ‡ |
| acid | | | | | | |
| Succinic acid | In | In | In/Di | In | In | In |
| Sulfuric acid | 1 ‡ | 1 ‡ | 2 Ω | In | IN | 2 ‡ |
| Thiocyanic acid | In | In | Po | In | In | 1 ‡ |

‡ Crystalline
Ω Poorly Crystalline
In Input material
Di Dioxane solvate
Th THF solvate
Ac Free Acid
Numbers represent the different XRPD patterns for each acid;
An asterisk (*) indicates that the solid was obtained from evaporation.

The acid salt screen produced fewer novel crystalline XRPD patterns than the base salt screen. Acids with a lower pKa tended to form salts more than those with a higher pKa. The formation of both THF and 1,4-dioxane solvates of sulfasalazine was observed, whereas it was not observed in the base salt screen. The solubility screen demonstrates that without the presence of a counterion, THF and 1,4-dioxane solvates would form.

The benzenesulfonic acid salt was chosen for scale up due to its having desirable thermal properties. The minimal mass loss due to decomposition (0.0%) showed that the sulfasalazine benzenesulfonic acid salt was easily dried and was not a solvated form. Furthermore, the high melting point (193° C.) demonstrated that this salt was stable in the solid state.

Secondary Assessment of Benzenesulfonic acid Salt

The table below (Table 11) summarizes the results of the secondary assessment of the benzenesulfonic acid salt ofsulfasalazine:

TABLE 11
Summary of the Results of the Secondary Assessment

| | | Analytical | |
|---|---|---|---|
| | | Technique | Benzenesulfonic Acid |
| Basic | | XRPD | Crystalline |
| Characterization | | TG/DTA | 0.3% loss up to degradation |
| | | DSC | Endotherms at 196 and 204° C. |
| | | ¹H NMR | 1:1 salt, minimal solvent |
| | | HPLC | 98.1% |
| | | DVS | Increase of 0.7% at 90% RH |
| 1-Week stress | 40° C., | XRPD | Unchanged |
| test | 75% RH | HPLC | 97.3% |
| | 80° C. | XRPD | Unchanged |
| | | HPLC | 98.1% |
| | Ambient | XRPD | Unchanged |
| | Light | HPLC | 98.1% |
| Salt Disproportionation | | XRPD | Sulfasalazine |
| Hydration | Low | XRPD | Partially Sulfasalazine |
| Studies | Medium | XRPD | Partially Sulfasalazine |
| | High | XRPD | Sulfasalazine |
| Thermodynamic | pH 1 | XRPD | Sulfasalazine |
| solubility | | Concentration | <LOD |
| | pH 4.5 | XRPD | Sulfasalazine |
| | | Concentration | <LOD |
| | pH 6.8 | XRPD | Sulfasalazine |
| | | Concentration | <LOD |

### Discussion

Sulfasalazine is a free base that exists in a crystalline form but has poor solubility in most solvents. A salt screen was carried out on the compound using 24 acidic counterions in 6 solvent systems. Each experiment was temperature cycled to encourage salt formation, and if no solids were present, the solvent was evaporated. Solids with unique XRPD patterns were analyzed by TG/DTA to assess their thermal properties, and ¹H NMR and stability assessments were also performed on some salts.

The benzenesulfonic acid salt was selected for a secondary salt screen where it was made on a 500 mg scale to be further analyzed. The salt from benzenesulfonic acid scaled up well to give a solid with an XRPD pattern that was consistent with the solid from the primary screen. The salt had excellent thermal (0.3% mass loss, 1919, 204° C. melts) and DVS (0.7% mass uptake) properties and did not change under stability stress conditions. Overall, the benzenesulfonic acid salt had desirable solid state properties.

### Example 2

### Screening Bases for Acid Salts of Sulfasalazine

The subject salt forms of sulfasalazine can provide higher apparent solubility than sulfasalazine. Other properties that can be improved are crystallinity and physical form stability. Such salts forms can find use as API in pharmaceutical compositions.

Salt screening was performed to identify particular crystalline and developable salt forms of sulfasalazine. The screening produced a number of pharmaceutically acceptable salts which have suitable physical properties for further development, including some forms with significantly higher aqueous solubility compared to the free acid form of sulfasalazine.

### Initial Characterization

On receipt of sulfasalazine, initial characterization was performed using XRPD, PLM, TG/DTA, DSC, DVS, 1H NMR, UPLC and LC-MS.

### Primary Salt Screen

The primary salt screen was carried out using stock solutions of 16 bases (1M), which were prepared in water. These were added to the free acid sulfasalazine suspended in a selection of six solvents. The procedure is described below. The bases and solvents used in the study can be found in Table 12 and Table 13 respectively.
Approximately 50 mg of sulfasalazine was weighed into each of 96 vials;
1 mL of the appropriate solvent was added to form a suspension of the solid;
131.8 μL of the appropriate base stock solution (1.05 equivalents) was added to each vial;
The resulting samples were then temperature cycled between ambient and 40° C. in 4 hour cycles for ca. 72 hours. Where material amounts were insufficient, the solvent was allowed to evaporate to attempt to recover solids;
Solids produced were analyzed by XRPD, TG/DTA and 1H NMR where material amounts allowed. Furthermore, stability studies at 40° C./75% RH were carried out on solids for ca. 48 hours.

TABLE 12
List of Primary Salt Screen Bases

| | Base |
|---|---|
| 1 | 1-(2-Hydroxyethyl)-pyrrolidine |
| 2 | Ammonium hydroxide |
| 3 | Benzathine |
| 4 | Choline |
| 5 | Diethanolamine |
| 6 | Diethylamine |
| 7 | Deanol |
| 8 | Hydroxyethyl morpholine |
| 9 | L-Arginine |
| 10 | L-Lysine |
| 11 | Meglumine |
| 12 | Piperazine |
| 13 | Potassium hydroxide |
| 14 | Sodium hydroxide |
| 15 | Triethanolamine |
| 16 | Tromethamine |

TABLE 13
List of Primary Salt Screen Solvents

| | | ICH |
|---|---|---|
| | Solvent | Class |
| 1 | Acetone | 3 |
| 2 | Acetonitrile | 2 |
| 2 | Dioxane | 2 |
| 4 | Ethanol | 3 |
| 5 | IPA | 3 |
| 6 | THF | 2 |

### Secondary Salt Screen and Developability Assessment

After the primary salt screen, four salts were put forward for scale up. The salts were produced on a 500 mg scale using the following procedure:
Approximately 500 mg of sulfasalazine was accurately weighed into a 20 mL scintillation vial;
10 mL of the appropriate solvent was added to the solid to create a suspension;
Stock solutions of each base were prepared to 1M concentration in water;
A volume of the appropriate stock solution (1318 μL, 1.05 equivalents) was added to each suspension;
The vials were then shaken for ca. 72 hours while temperature cycling between ambient and 40° C. in 4 hour cycles;
For the vials in which there was solid remaining, this was filtered off and dried under vacuum at ambient temperature for ca. 3 hours;
For vials in which there was no solid, the vials were uncapped and left until the solvent had evaporated. Once the solvent had evaporated, the resulting solid was dried under vacuum at ambient temperature for ca. 3 hours;
   A yield of each experiment was calculated and the solids were analyzed according to the same methods as detailed above in Example 1.

### Results

### Initial Characterization

Initial characterization of sulfasalazine indicated the following:
By XRPD, the sample was crystalline with a small amorphous content;
PLM images showed sulfasalazine to be made of small, birefringent particles;
The TGA showed minimal weight loss up to degradation at ca. 260° C. A single endothermic event was observed in the DTA, with an onset temperature of ca. 259° C.;
In the first heating cycle, the DSC showed an endothermic event with two peaks (onset temperatures of ca. 236 and 246° C.). The cooling cycle showed a small event with onset at ca. 45° C. This is likely to be a glass transition as the amorphous solid cools from the melt. No events were observed in the second heating cycle;
By DVS, sulfasalazine was minimally hygroscopic with a mass uptake of ca. 1.0% at 90% RH. There was no evidence of a form change during experimentation and the XRPD pattern was retained after being exposed to DVS humidity;
The ¹H NMR spectrum of sulfasalazine had 11 protons in the aromatic region, consistent with the structure;
The purity of the sample by UPLC was found to be 96.4%;
The molecular ion peak in the mass spectrum was at 399.05 m/z, consistent with the M+H value of the compound.

### pKa Determination

The pKa (Table 14) and log P (Table 15) values of sulfasalazine are detailed below. The molecule has two acidic groups, with pKa values of 2.29 and 10.96, and one basic group, with a pKa value of 8.05. The fact that sulfasalazine can exist in either the cationic or zwitterionic forms means that it has two log P values, one for each form.

TABLE 14
pKa Values of Sulfasalazine

| | | | Ionic | |
|---|---|---|---|---|
| pKₐ | Type* | T/° C. | Environment | Method |
| 2.29 ± 0.01 | Acid | 25.0 | 0.15M KCl | UV-metric |
| 8.05 ± 0.01 | Base | 25.0-25.1 | 0.15M KCl | UV-metric |
| 10.96 ± 0.02 | Acid | 25.0-25.1 | 0.15M KCl | UV-metric |

TABLE 15
logP Values of Sulfasalazine

| | | | Ionic | |
|---|---|---|---|---|
| LogP | Species | T/° C. | Environment | Method |
| 3.73 ± 0.01 | Cationic | 24.9-25.0 | 0.15M KCl | pH-metric |
| 0.02 ± 0.03 | Zwitterionic | 24.9-25.0 | 0.15M KCl | pH-metric |

### Primary Salt Screen

The primary salt screen was carried out with 16 bases in 6 solvents as described above. Solids with unique crystalline XRPD patterns were analyzed by TG/DTA and also by ¹H NMR where material amounts allowed. Where multiple solids had the same pattern, the most crystalline solid by XRPD was analyzed.

1. 1-(2-Hydroxyethyl)-pyrrolidine

The primary salt screen with 1-(2-hydroxyethyl)-pyrrolidine returned:
No solids after temperature cycling;
Six solids after evaporation;
   Four crystalline solids from acetone, acetonitrile, 1,4-dioxane and THF;
   Two amorphous solids from ethanol and 2-propanol.

In total, two distinct crystalline XRPD patterns were observed, one from acetone and acetonitrile and a second from 1,4-dioxane and THF.

### 2. Ammonium Hydroxide

The primary salt screen with ammonium hydroxide returned:
One solid after temperature cycling;
   Crystalline solid from acetonitrile;
   Insufficient material from all other solvent systems;
Four solids after evaporation;
   Three crystalline solids from 1,4-dioxane, ethanol and 2-propanol;
   One amorphous solid from acetone;
   Insufficient material from THF

In total, four distinct crystalline XRPD patterns were observed. Three of the solids were analyzed by TG/DTA.

### 3. Benzathine (N,N′-Dibenzylethylenediamine)

The primary salt screen with benzathine returned:
Four solids after temperature cycling;
   Crystalline solid from acetonitrile, 1,4-dioxane, ethanol and 2-propanol;
   Insufficient material from all other solvent systems;
Two solids after evaporation;
   One crystalline solid from acetone;
   One mostly amorphous solid from THF;

In total, four distinct crystalline XRPD patterns were observed. Three of the solids were analyzed by TG/DTA.

### 4. Choline Hydroxide

The primary salt screen with choline hydroxide returned:
No solids after temperature cycling;
No solids after evaporation.

### 5. Diethanolamine

The primary salt screen with diethanolamine returned:
No solids after temperature cycling;
Four solids after evaporation;
   Four crystalline solids from acetone, acetonitrile, ethanol and THF;
   Insufficient material from 1,4-dioxane and 2-propanol.
      In total, two distinct XRPD patterns were observed.

### 6. Diethylamine

The primary salt screen with diethylamine returned:
No solids after temperature cycling;
Six solids after evaporation;
   Six crystalline solids from all solvent systems were analyzed.

In total, three distinct XRPD patterns were observed—one from acetone, acetonitrile, ethanol and 2-propanol, one from 1,4-dioxane and one from THF. Although it is likely that, due to the similarity of the diffractograms, all solids contain some of the same solid form, the additional peaks in the pattern from 1,4-dioxane THF suggest the presence of an additional form. One of the solids was analyzed by TG/DTA. One solid was subjected to stability stress test conditions of 40° C./75% RH for ca. 48 hours.

### 7. Deanol (Dimethylaminoethanol)

The primary salt screen with deanol returned:
No solids after temperature cycling;
Three solids after evaporation;
   Two crystalline solids from ethanol and THF;
   One amorphous solid from 2-propanol.

In total, two distinct XRPD patterns were observed. Although there are some similarities between the two diffractograms, the presence of additional peaks in the solid from THF suggest the presence of an additional form. One of the solids was analyzed by TG/DTA.

### 8. Hydroxyethylmorpholine

The primary salt screen with hydroxyethylmorpholine returned:
One solid after temperature cycling;
   One amorphous solid from 2-propanol.
No solids after evaporation.

### 9. L-Arginine

The primary salt screen with L-arginine returned:
Five solids after temperature cycling (FIG. 33); ∘ Three crystalline solids from acetone, acetonitrile and 1,4-dioxane;
   One mostly amorphous solid from ethanol;
   One amorphous solid from 2-propanol;
One solid after evaporation;
   One mostly amorphous solid from THF.

In total, three distinct crystalline XRPD patterns were observed. Two of the solids were analyzed by TG/DTA. Two solids were subjected to stability stress test conditions of 40° C./75% RH for ca. 48 hours:
The solid from acetonitrile had the same XRPD pattern after the test;
The solid from 1,4-dioxane became amorphous after the test.

### 10. L-Lysine

The primary salt screen with L-lysine returned:
Five solids after temperature cycling. The same patterns were observed and confirmed using a logarithmic counts scale to show that the peaks occur in the same position;
   Crystalline solid from acetone, acetonitrile, 1,4-dioxane, ethanol and 2-propanol;
   Insufficient material from THF;
One solid after evaporation;
   One crystalline solid from THF.

In total, one distinct crystalline XRPD pattern was observed, although the pattern from THF had an extra peak at low 20. One of the solids was analyzed by TG/DTA:
The solid from 1,4-dioxane had a mass loss of ca. 2.2% up to decomposition. At ca. 217° C., a small melt was followed immediately by an exothermic decomposition.

One solid was subjected to stability stress test conditions of 40° C./75% RH for ca. 48 hours: the solid from 1,4-dioxane had the same XRPD pattern after the test. One solid was analyzed by 1H NMR.

### 11. Meglumine

The primary salt screen with meglumine returned:
Two solids after temperature cycling;
   Amorphous material from acetonitrile and 2-propanol;
   Insufficient material from all other solvent systems;
Three solids after evaporation;
   Three partially crystalline solids from acetone, ethanol and THF;
   Insufficient material from 1,4-dioxane.

In total, one distinct crystalline XRPD patterns was observed.

### 12. Piperazine

The primary salt screen with piperazine returned:
Six solids after temperature cycling;
   Crystalline solid from acetone, acetonitrile, 1,4-dioxane, ethanol and 2-propanol;
   Mostly amorphous material from THF;

In total, one distinct crystalline XRPD patterns was observed. One of the solids was analyzed by TG/DTA and one solid was subjected to stability stress test conditions of 40° C./75% RH for ca. 48 hours: the solid from acetonitrile had the same XRPD pattern after the test;

### 13. Potassium Hydroxide

The primary salt screen with potassium hydroxide returned:
Three solids after temperature cycling;
   Crystalline solid from acetonitrile, ethanol and 2-propanol;
   Insufficient material from all other solvent systems;
Three solids after evaporation;
   Crystalline solid from acetone, 1,4-dioxane and THF.

In total, six distinct crystalline from each solvent system produced a different XRPD pattern. Three of the solids were analyzed by TG/DTA. Three solids were subjected to stability stress test conditions of 40° C./75% RH for ca. 48 hours:
The solid from acetonitrile had a different XRPD pattern after the test;
The solid from ethanol had the same XRPD pattern after the test;
The solid from 2-propanol had the same XRPD pattern after the test.

### 14. Sodium Hydroxide

The primary salt screen with sodium hydroxide returned:
Two solids after temperature cycling;
   Crystalline solid from acetonitrile and 2-propanol;
   Insufficient material from all other solvent systems;
Three solids after evaporation;
   Crystalline solid from acetone, ethanol and THF;
   Insufficient material from 1,4-dioxane.

In total, four distinct crystalline XRPD patterns were observed—one from acetone, one from acetonitrile, one from ethanol and 2-propanol and one from THF. Two of the solids were analyzed by TG/DTA and two solids were subjected to stability stress test conditions of 40° C./75% RH for ca. 48 hours:
The solid from acetonitrile had a different XRPD pattern after the test;
The solid from 2-propanol had a different XRPD pattern after the test.

### 15. Triethanolamine

The primary salt screen with triethanolamine returned:
Two solids after temperature cycling;
   One crystalline solid from ethanol;
   One amorphous solid from 2-propanol;
   Insufficient material from all other solvent systems;
Four solids after evaporation;
   Four crystalline solids from acetone, acetonitrile, 1,4-dioxane and THF;

In total, two distinct crystalline XRPD patterns were observed that were different from the input material—one from acetone, 1,4-dioxane and THF and a second from acetonitrile. One of the solids was analyzed by TG/DTA and one solid was subjected to stability stress test conditions of 40° C./75% RH for ca. 48 hours:
The solid from acetone had the same XRPD pattern after the test;

### 16. Tromethamine

The primary salt screen with tromethamine returned:
One solid after temperature cycling;
   Amorphous solid from acetonitrile;
   Insufficient material from all other solvent systems;
Four solids after evaporation;
   Three crystalline solids from acetone, ethanol and THF;
   One partially crystalline solid from 2-propanol;
   Insufficient material from 1,4-dioxane.

In total, one distinct crystalline XRPD pattern was observed. One of the solids was analyzed by TG/DTA and three solids were subjected to stability stress test conditions of 40° C./75% RH for ca. 48 hours:
The solid from ethanol had an XRPD pattern had reduced crystallinity after the test.

### Secondary Salt Screen and Developability Assessment

After the primary salt screen, the following four salts were selected for the secondary screen (Table 16):

TABLE 16
List of the Salts Selected for Scale Up

| | Counterion | Solvent |
|---|---|---|
| 1 | Diethylamine | Ethanol |
| 2 | L-Lysine | Acetone |
| 3 | Triethanolamine | Acetone |
| 4 | Tromethamine | Ethanol |

The results from the scale up of the diethylamine, L-lysine, triethanolamine and tromethamine salts are detailed below:

1. Diethylamine from Ethanol
XRPD analysis found the scaled up solid to have a similar XRPD pattern to the solid from the primary screen but with an additional peak at ca. 6° 2θ. There was reduced crystallinity of the solid with scale up;
By TGA the solid had a mass loss of ca. 1.1% before a large two-stage mass loss, which could both be decomposition or the initial loss could be desolvation. Analysis of the ¹H NMR spectrum suggests that it could be loss of water. A melting event was observed in the DTA before decomposition at ca. 182° C.;
There was a single endothermic event in the DSC thermogram for the first heating cycle, with onset temperature ca. 191° C., agreeing with the DTA. There were no events in the cooling or second heating cycles;
The DVS isotherm plot showed the solid to be minimally hygroscopic, with a mass uptake of ca. 1.0% at 90% RH. In the kinetic plot, no obvious change in solid form occurred. After DVS analysis, the XRPD pattern of the solid remained unchanged;
An IR spectrum of the scaled up solid was taken for reference. A broad peak corresponding to the presence of an —OH group could be seen at ca. 2750 cm⁻¹;
¹H NMR analysis showed the salt to be a 1:1 ratio of acid to base. Only 0.05 equivalents of the solvent, ethanol, were present;
After 1-week stability studies on the solid the following results were found (FIG. 4):
   40° C./75% RH—the XRPD pattern was unchanged and the purity (Table 17) was also unchanged (97.6%);
   80° C.—the XRPD pattern was slightly different after stability studies, with the peak at ca. 6° 2θ being lost. The purity of the sample had increased to 98.3%;
   Ambient light—the XRPD pattern was slightly different after stability studies, with the peak at ca. 6° 2θ being lost. The purity was the same as the input at 97.6%;
The salt had a purity of 97.6% by HPLC;
After hydration studies, the following results were found, and pH values are in Table 18:
   Low water activity—there were slight changes in the XRPD pattern, for example the loss of the small peak at 6° 2θ;
   Medium water activity—there were slight changes in the XRPD pattern, for example the loss of the small peak at 6° 2θ;
   High water activity—no solid could be obtained due to complete dissolution;
After thermodynamic solubility studies, the following results were found, and pH values are in Table 19:
   pH 1 buffer—by XRPD, the solid had reverted to sulfasalazine. The amount of solid in solution was less than the limit of detection;
   pH 4.5 buffer—there were small changes to the XRPD pattern, for example the loss of the small peak at 6° 2θ and there was a concentration of 1.7 mg·mL-1 (Table 20);
pH 6.8 buffer—there were small changes to the XRPD pattern, for example the loss of the small peak at 6° 2θ. The concentration of the solution was 7.7 mg·mL⁻¹.

TABLE 17
UPLC Purity Values of the Scaled Up Diethylamine
Salt After Stability Studies

| | Average |
|---|---|
| Condition | Purity |
| 40° C./75% RH | 97.6 |
| 80° C. | 98.3 |
| Ambient light | 97.6 |

TABLE 18
pH Values of the Scaled Up Diethylamine
Salt Pre- and Post-Hydration Studies

| Water | Pre-agitation | Post-agitation |
|---|---|---|
| Activity | pH | pH |
| 0.281 | 5.52 | 5.11 |
| 0.572 | 5.71 | 5.35 |
| 0.790 | 5.40 | 5.29 |

TABLE 19
pH Values of the Scaled Up Diethylamine Salt Pre-
and Post-Thermodynamic Solubility Studies

| Buffer | Pre-agitation | Post-agitation |
|---|---|---|
| pH | pH | pH |
| 1 | 0.57 | 1.52 |
| 4.5 | 5.22 | 6.66 |
| 6.8 | 6.72 | 6.98 |

TABLE 20
Thermodynamic Concentration Values
of the Scaled Up Diethylamine Salt

| Buffer | Concentration |
|---|---|
| pH | (mg · mL⁻¹) |
| 1 | Less than limit |
| | of detection |
| 4.5 | 1.7 |
| 6.8 | 7.7 |

2. L-Lysine from Acetone
XRPD analysis found the scaled up solid to have the same XRPD pattern as the solid from the primary screen. The differences in peak intensity can be attributed to the presence of preferential orientation in the primary screen solid;
By TGA the solid had a mass loss of ca. 3.0% up to the exothermic decomposition at ca. 222° C. No events were observed in the DTA before decomposition;
There were no events in the DSC thermogram for the first heating cycle, agreeing with the DTA. In the cooling cycle, a small endothermic event with onset temperature ca. 86° C. was observed, which could be due to a glass transition. No events were observed in the second heating cycle;
The DVS isotherm plot showed the solid to be moderately hygroscopic, with a mass uptake of ca. 4.9% at 90% RH. In the kinetic plot, no obvious change in solid form occurred. After DVS analysis, the XRPD pattern of the solid remained unchanged;
An IR spectrum of the scaled up solid was taken for reference. A broad peak corresponding to the presence of an —OH group could be seen at ca. 2950 cm⁻¹;
¹H NMR analysis showed the salt to be a 1:1 ratio of acid to base;
The salt had a purity of 98.2% by HPLC;
After 1-week stability studies on the solid the following results were found (FIG. 5):
   40° C./75% RH—the XRPD pattern was slightly different after stability studies, with an extra peak forming at ca. 6.5° 2θ. The purity was approximately the same (Table 21, 98.3%);
80° C.—the XRPD pattern was slightly different after stability studies, with an extra peak forming at ca. 6.5° 20. The purity was approximately unchanged at 98.4%;
Ambient light—the XRPD pattern was retained and the purity was unchanged (98.1%);
After hydration studies, the following results were found, and pH values are in Table 22:
   Low water activity—the XRPD pattern was unchanged;
   Medium water activity—there were slight changes in the XRPD pattern;
   High water activity—there were slight changes in the XRPD pattern;
After thermodynamic solubility studies, the following results were found, and pH values are in Table 23:
   pH 1 buffer—by XRPD, the solid had reverted to sulfasalazine. The amount of solid in solution was less than the limit of detection;
   pH 4.5 buffer—there were some changes to the XRPD pattern, for example additional peaks at around 8° 2θ. The concentration of the solution was 1.7 mg·mL⁻¹ (Table 24);
   pH 6.8 buffer—there were some changes to the XRPD pattern, for example the large peak at 18° 20. The concentration of the solution was 6.7 mg·mL⁻¹.

TABLE 21
UPLC Purity Values of the Scaled Up
L-Lysine Salt After Stability Studies

| | Average |
|---|---|
| Conditions | Purity |
| 40° C./75% RH | 98.3 |
| 80° C. | 93.4 |
| Ambient light | 98.1 |

TABLE 22
pH Values of the Scaled Up L-Lysine
Salt Pre- and Post-Hydration Studies

| Water | Pre-agitation | Post-agitation |
|---|---|---|
| Activity | pH | pH |
| 0.281 | 6.53 | 6.52 |
| 0.572 | 6.77 | 7.55 |
| 0.790 | 8.28 | 7.90 |

TABLE 23
pH Values of the Scaled Up L-Lysine Salt Pre-
and Post-Thermodynamic Solubility Studies

| Buffer | Pre-agitation | Post-agitation |
|---|---|---|
| pH | pH | pH |
| 1 | 0.76 | 1.91 |
| 4.5 | 4.70 | 5.63 |
| 6.8 | 6.99 | 7.17 |

TABLE 24
Thermodynamic Solubility Values of Scaled Up L-Lysine Solid

| Buffer | Concentration |
|---|---|
| pH | (mg · mL⁻¹) |
| 1 | Less than limit |
| | of detection |
| 4.5 | 1.7 |
| 6.8 | 6.7 |

3. Triethanolamine from Acetone
XRPD analysis found the scaled up solid to have the same XRPD pattern as the solid from the primary screen. There was improved crystallinity of the solid with scale up;
By TGA the solid had a mass loss of ca. 1.7% up to the exothermic decomposition at ca. 205° C. A melting event was observed in the DTA before decomposition at ca. 156° C.;
There was a single endothermic event in the DSC thermogram for the first heating cycle, with onset temperature ca. 154° C., agreeing with the DTA. In the cooling cycle, a large endothermic event with onset temperature ca. 206° C. was observed, which could be due to crystallization of the solid from the melt. No events were observed in the second heating cycle, possibly due to some decomposition of the solid;
The DVS isotherm plot showed the solid to be moderately hygroscopic, with a mass uptake of ca. 3.9% at 90% RH. In the kinetic plot, no obvious change in solid form occurred. After DVS analysis, the XRPD pattern of the solid remained unchanged;
An IR spectrum of the scaled up solid was taken for reference. A broad peak corresponding to the presence of an —OH group could be seen at ca. 3000 cm⁻¹;
¹H NMR analysis showed the salt to be a 1:1 ratio of acid to base. Only trace amounts of the solvent, acetone, were present;
The salt had a purity of 97.3% by HPLC;
After 1-week stability studies on the solid the following results were found (FIG. 6):
   40° C./75% RH the XRPD pattern was retained and the purity was approximately the same (Table 25 97.5%);
80° C.—there were difference in peak intensity compared to the pattern before the study. The purity of the solid was unchanged (97.3%);
Ambient light—the XRPD pattern was retained, as was the purity (97.4%);
After thermodynamic solubility studies, the following results were found, and pH values are in Table 26: ∘ pH 1 buffer—by XRPD, the solid had reverted to sulfasalazine. There was insufficient material for solubility to be determined;
   pH 4.5 buffer—the solid was amorphous by XRPD, although the peaks that remained were consistent with the input material. This could be due to the limited material available for analysis. The concentration of the solution was 0.1 mg·mL-1 (Table 27);
   pH 6.8 buffer—the solid was amorphous by XRPD, although the peaks that remained were consistent with the input material. This could be due to the limited material available for analysis. The concentration of the solution was 0.9 mg·mL⁻¹.

TABLE 25
UPLC Purity Values of the Scaled Up Triethanolamine
Salt After Stability Studies

| | Average |
|---|---|
| Condition | Purity |
| 40° C./75% RH | 97.5 |
| 80° C. | 97.3 |
| Ambient light | 97.4 |

TABLE 26
pH Values of the Scaled Up Triethanolamine Salt
Pre- and Post- Thermodynamic Solubility Studies

| Buffer | Pre-agitation | Post-agitation |
|---|---|---|
| pH | pH | pH |
| 1 | 1.25 | 1.54 |
| 4.5 | 5.97 | 6.15 |
| 6.8 | 6.76 | 7.38 |

TABLE 27
Thermodynamic Solubility Values
of Scaled Up Triethanolamine Salt

| Buffer | Concentration |
|---|---|
| pH | (mg · mL⁻¹) |
| 1 | Insufficient |
| | material |
| 4.5 | 0.1 |
| 6.8 | 0.9 |

4. Tromethamine from Ethanol
XRPD analysis found the scaled up solid to have the same XRPD pattern as the solid from the primary screen;
By TGA the solid had a mass loss of ca. 3.0% up to the exothermic decomposition at ca. 252° C.). A melting event was observed in the DTA before decomposition with an onset temperature of ca. 129° C.;
There were two endothermic events in the DSC thermogram for the first heating cycle, with onset temperatures ca. 67 and 123° C. The latter of these two events agrees with the event in the DTA, however the smaller event was not observed in the DTA There were no events in the cooling or second heating cycles;
The DVS isotherm plot showed the solid to be moderately hygroscopic, with a mass uptake of ca. 8.9% at 90% RH. In the kinetic plot, no obvious change in solid form occurred. After DVS analysis, the XRPD pattern of the solid remained unchanged;
An IR spectrum of the scaled up solid was taken for reference. A broad peak corresponding to the presence of an —OH group could be seen at ca. 3000 cm⁻¹;
¹H NMR analysis showed the salt to be a 1:1 ratio of acid to base. Only 0.04 equivalents of the solvent, ethanol, were present;
The salt had a purity of 97.5% by HPLC;
After 1-week stability studies on the solid the following results were found (FIG. 7):
   40° C./75% RH—the XRPD pattern was the same as that of the input material, although there was some reduction in crystallinity The purity of the solid was unchanged (97.4%, Table 28);
80° C.—the XRPD pattern was consistent with that of the input material and the purity was slightly increased (97.8%);
   Ambient light—the XRPD pattern was consistent with that of the input material and there was a slight decrease in the purity (97.2%);
After thermodynamic solubility studies, the following results were found, and pH values are in Table 29:
   pH 1 buffer—the solid had a different pattern compared with the input material and had more amorphous content. The amount of solid in solution was less than the limit of detection;
   pH 4.5 buffer—the solid had a different pattern compared with the input material. The concentration of the solution was 0.3 mg·mL-1 (Table 30);
   pH 6.8 buffer—the solid had a different pattern compared with the input material, but the pattern was the same as that from pH 4.5 buffer. There was insufficient material left for solubility determination.

TABLE 28
UPLC Purity Values of the Scaled Up Tromethamine
Salt After Stability Studies

| | Average |
|---|---|
| Condition | Purity |
| 40° C./75% RH | 97.4 |
| 80° C. | 97.8 |
| Ambient light | 97.2 |

TABLE 29
pH Values of the Scaled Up Tromethamine Salt
After Thermodynamic Solubility Studies

| Buffer | Pre-agitation | Post-agitation |
|---|---|---|
| pH | pH | pH |
| 1 | 1.36 | 1.61 |
| 4.5 | 5.80 | 6.98 |
| 6.8 | 6.89 | 6.95 |

TABLE 30
Thermodynamic Solubility Values
of Scaled Up Tromethamine Salt

| Buffer | Concentration |
|---|---|
| pH | (mg · mL⁻¹) |
| 1 | Less than the limit |
| | of detection |
| 4.5 | 0.3 |
| 6.8 | Insufficient |
| | material |

5. Sulfasalazine

The original material was also subjected to the same solubility experiments as the salts in order to provide a reference. The results can be found in Table 31.

TABLE 31
Thermodynamic Solubility Values for Sulfasalazine

| Buffer | Concentration |
|---|---|
| pH | (mg · mL⁻¹) |
| 1 | Less than the limit |
| | of detection |
| 4.5 | Less than the limit |
| | of detection |
| 6.8 | Less than the limit |
| | of detection |

### SUMMARY OF RESULTS Primary Salt Screen

The table below (Table 32) summarizes the results of the primary base salt screen on sulfasalazine:

TABLE 32
Summary of Results of the Primary Salt Screen

| | Ace- | Aceto- | 1,4- | Etha- | 2- | |
|---|---|---|---|---|---|---|
| | tone | nitrile | Dioxane | nol | Propanol | THF |
| 1-(2-Hydroxyethyl)-pyrrolidine | *1 ‡ | *1 ‡ | *2 ‡ | *Ω | *Ω | *2 ‡ |
| Ammonium hydroxide | Ω | 1 ‡ | *2 ‡ | *3 ‡ | *4 ‡ | |
| Benzathine | *1 ‡ | 2 ‡ | 3 ‡ | 1 ‡ | 2 ‡ | *4 ‡ |
| Choline | | | | | | |
| Diethanolamine | *1 ‡ | *2 ‡ | | *2 ‡ | | *1 ‡ |
| Diethylamine | *1 ‡ | *1 ‡ | *2 ‡ | *1 € | *1 ‡ | *3 ‡ |
| Deanol | | | | *1 ‡ | *Ω | *2 ‡ |
| Hydroxyethyl morpholine | | | | | Ω | |
| L-Arginine | 1 ‡ | 2 ‡ | 3 ‡ | PD ‡ | Ω | *PD 4 ‡ |
| L-Lysine | PO 1 € | PO 1 ‡ | 1 ‡ | 1 ‡ | 1 ‡ | *2 ‡ |
| Meglumine | *PD 1 ‡ | Ω | | *PD 1 ‡ | Ω | *PD 1 ‡ |
| Piperazine | 1 ‡ | 1 ‡ | 1 ‡ | 1 ‡ | 1 ‡ | PD 1 ‡ |
| Potassium hydroxide | *1 ‡ | 2 ‡ | *3 ‡ | 4 ‡ | 5 ‡ | *6 ‡ |
| Sodium hydroxide | *1 ‡ | 2 ‡ | | *PD 3 ‡ | 3 ‡ | *4 ‡ |
| Triethanolamine | *1 € | *2 ‡ | *1 ‡ | FA ‡ | Ω | *1 ‡ |
| Tromethamine | *1 ‡ | Ω | | *1 € | *PD ‡ | *1 ‡ |

€ Scaled Up
‡ Crystalline
Ω Amorphous
PO Preferred Orientation
PD Poor Diffraction
Insufficient Material
Numbers represent the different XRPD patterns for each acid;
An asterisk (*) indicates that the solid was obtained from evaporation.

### Secondary Assessment of Salts of Interest

The table below (Table 33) summarizes the results of the secondary base salt screen on sulfasalazine:

TABLE 33
Summary of the Results of the Secondary Screen

| | | Analytical | | | | |
|---|---|---|---|---|---|---|
| | | technique | Diethylamine | L-Lysine | Triethanolamine | Tromethamine |
| Basic characterisation | | XRPD | Crystalline | Crystalline | Crystalline | Crystalline |
| | | TG/DTA | 1.1% loss up | 3.0% loss up | 1.7% loss up | 3.0% loss up |
| | | | to degradation | to degradation | to degradation | to degradation |
| | | DSC | Endotherm at | No events | Endotherm at | Endotherms at |
| | | | 191° C. | | 154° C. | 67 and 123° C. |
| | | ¹H NMR | 1:1 salt, | 1:1 salt, | 1:1 salt, | 1:1 salt, |
| | | | minimal | minimal | minimal | minimal |
| | | | solvent | solvent | solvent | solvent |
| | | HPLC | 97.6% | 98.2% | 97.3% | 97.5% |
| | | DVS | Increase | Increase | Increase | Increase |
| | | | of 1.0% at | of 5.0% at | of 3.9% at | of 8.9% at |
| | | | 90% RH | 90% RH | 90% RH | 90% RH |
| 1-Week | 40° C. | XRPD | Unchanged | Small change | Small change | Reduced |
| stress test | 75% RH | | | | | crystallinity |
| | | HPLC | 97.6% | 98.3 | 97.5 | 97.4 |
| | 80° C. | XRPD | Unchanged | Small change | Small change | Unchanged |
| | | HPLC | 98.3 | 98.4 | 97.3 | 97.8 |
| | Ambient | XRPD | Unchanged | Unchanged | Small change | Unchanged |
| | Light | HPLC | 97.6 | 98.1 | 97.4 | 97.2 |
| Salt disproportionation | | XPRD | Small change | New peak | Amorphous | Amorphous |
| Hydration | Low | XRPD | Small change | Unchanged | Small change | No solid |
| studies | Medium | XRPD | Small change | Small change | Small change | No solid |
| | High | XRPD | No solid | Small change | No solid | No solid |
| Thermo- | pH 1 | XRPD | Sulfasalazine | Sulfasalazine | Sulfasalazine | Unchanged |
| dynamic | | Concen- | <LOD | <LOD | | <LOD |
| solubility | | tration | | | | |
| | pH 4.5 | XRPD | Small change | Small change | | Changed |
| | | Concen- | 1.7 mg · mL⁻¹ | 1.7 mg · mL⁻ | 0.1 mg · mL⁻ | 0.3 mg · mL⁻ |
| | | tration | | | | |
| | pH 6.8 | XRPD | Small change | Small change | Amorphous | Changed |
| | | Concen- | 7.7 mg · mL⁻¹ | 6.7 mg · mL⁻ | 0.9 mg · mL⁻ | Insufficient |
| | | tration | | | | material |

### Discussion

Sulfasalazine is a free acid that can exist in a crystalline form but has poor solubility in most solvents. A salt screen was carried out on the compound using 16 basic counterions in 6 solvent systems. Each experiment was temperature cycled to encourage salt formation, and if no solids were present, the solvent was allowed to evaporate. Solids with unique XRPD patterns were analyzed by TG/DTA to assess their thermal properties, and, ¹H NMR and stability assessments were performed.

Four solids of interest were selected for the secondary salt screen stage where they were made on a 500 mg scale so that they could be fully analyzed. Diethylamine, L-lysine, triethanolamine and tromethamine salts were scaled up.

The salt from diethylamine scaled up well to give a solid with a XRPD pattern that was consistent with the primary screen but with an extra peak. The salt offered desirable overall thermal (1.1% mass loss, 191° C. melt) and DVS (1.0% mass uptake) properties, and did not change under stability stress conditions. The salt also demonstrates an improvement on solubility compared with sulfasalazine, especially at pH 6.8 (7.7 mg·mL⁻¹).

The salt from L-lysine also scaled up well, retaining the XRPD pattern seen in the primary screen. The solid was moderately hygroscopic (5.0% mass uptake), but had good thermal properties and was observed to form in multiple solvent systems. The salt also demonstrates an improvement on solubility compared with sulfasalazine, especially at pH 6.8 (6.7 mg·mL⁻¹).

The salt from triethanolamine had an XRPD pattern that was consistent with the solid from the primary screen. It was moderately hygroscopic (3.9% mass uptake) and had good thermal properties. This salt showed some changes under stability, disproportionation, hydration and solubility studies.

The salt from tromethamine scaled up well. This salt had the following thermal properties (3.0% mass loss, 67 and 123° C. events) and DVS properties (8.9% mass uptake).

Of these four salts of sulfasalazine that were scaled up, all would be developable.

### Example 3

### Solubilities of Sulfasalazine Salts

Solubility data of sulfasalazine diethylamine salt and sulfasalazine tromethamine salt was obtained for use in crystallization studies and screening for a stable polymorph. The salts aree insoluble in many solvents which provides for a large choice of antisolvents. The diethylamine salt has high solubility in polar aprotic solvents which could be used to obtain a homogeneous solution prior to crystallization of the sulfasalazine salt.

TABLE 34
Estimated Solubilities of Sulfasalazine Diethylamine Salt

| | Diethylamine Salt | Free Acid |
|---|---|---|
| | Solubility Estimate | Solubility |
| Solvent | (mg/mL) | (mg/mL) |
| acetone | 2 | 1.1 |
| acetonitrile (ACN) | <1 | 0.3 |
| 2-butanol | <1 | |
| chloroform | <1 | |
| EtOH | 3 | 0.5 |
| EtOAc | <1 | 0.4 |
| heptane | <1 | |
| isopropyl alcohol (IPA) | <1 | 0.5 |
| methyl tert-butyl ether (MTBE) | <1 | 0.3 |
| MeOH | 6 | 0.9 |
| methyl ethyl ketone (MEK) | 1 | 0.9 |
| THF | 6 | |
| water | 3 | 0.4 |

TABLE 35
Estimated Solubilities of Sulfasalazine Tromethamine Salt

| | Tromethamine Salt |
|---|---|
| | Solubility Estimate |
| Solvent | (mg/mL) |
| acetone | 2 |
| acetonitrile (ACN) | <1 |
| 2-butanol | <1 |
| chloroform | <1 |
| DMSO | >82 |
| EtOH | 5 |
| EtOAc | <1 |
| heptane | <1 |
| isopropyl alcohol (IPA) | <1 |
| MeOH | 27 |
| THF | 2 |
| water | 13 |

### Example 4

### Characterization of Sulfasalazine Salts

Sulfasalazine Diethylamine Salt

The sulfasalazine diethylamine salt Form A is crystalline with individual particle sizes ˜1-5 μm and agglomerates of ˜10-40 μm. The crystalline material has been designated as Form A.

Other polymorph forms of sulfasalazine diethylamine salt (Forms B-E) were also identified and characterized as part of these studies. Diethylamine salt Form A and Form B are anhydrous. Diethylamine salt Form C is a likely THF solvate. Diethylamine salt Form E is a likely hydrated form.

By TGA the solid Form A had a main endothermic event at ˜207° C. and a minor shoulder at ˜200° C. Decomposition likely occurs at the melt or immediately after. Diethylamine salt Form A is slightly hygroscopic, absorbing more than 1 wt % moisture at 95% RH. This crystalline form of the diethylamine salt is significantly less hygroscopic than the tromethamine salt.

Stability studies were performed by adapting the methods described herein. In one stability study, the diethylamine salt Form A was observed to be more stable that other diethylamine salt polymorphs at 50° C./75% RH and showed no evidence of degradation at t=5 days.

Sulfasalazine Tromethamine Salt

A crystalline sulfasalazine tromethamine (1:1) salt form (Form A) was prepared by adapting the method described above, characterized and assessed for stability. Form A is anhydrous and un-solvated. Other polymorph forms of sulfasalazine tromethamine salt (Forms B-G) were also identified and characterized as part of these studies. Polymorphs Forms B-G included solvates.

By TGA the solid Form A had an endotherm at ˜193° C. that is likely associated with the melt of the salt with decomposition immediately following. In addition, a small endothermic event at ˜141° C. may be due to the small presence of Form G polymorph melting. The tromethamine salt Form A is hygroscopic, absorbing over 8 wt % in moisture up to 95% RH.

Stability studies were performed by adapting the methods described herein. The sulfasalazine tromethamine (1:1) salt Form A appears to be equally stable as diethylamine salt Form A at 50° C./75% RH at t=5 days.

### Example 5

### Crystallization of Sulfasalazine Salts

A process was developed for the manufacture of 1-5 kg of a stable polymorph of sulfasalazine diethylamine salt (1:1). The solvent system used in this process for crystallization of the salt was 2-butanol/DMSO.

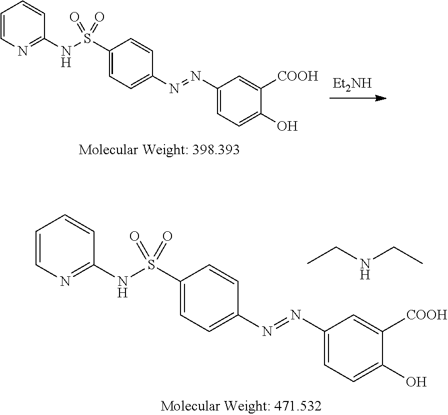

Exemplary process for preparation of crystalline sulfasalazine diethylamine salt:

1. Charge 1.0 kg sulfasalazine to a smaller reactor (R1).

2. Charge 1.50 L DMSO to R1.

3. Charge 0.285 L diethylamine to R1.
a. Noticed an exotherm at this point where the internal temp was at 36-37° C. (700 g scale)

4. Stir until complete dissolution.
a. Note the solution is dark red and it will be difficult to see when all material is dissolved.
b. Recommend stirring for 30-60 minutes to ensure complete dissolution.

5. Charge 0.50 L 2-BuOH to R1.
a. Total volume at his point is approximately 3-3.5 V (3-3.5 L)

6. Continue stirring for 5-10 minutes.

7. Polish filter this viscous solution through a polish filter into a larger reactor (R2).
a. Recommend 10 μm filter.

8. Rinse the smaller reactor (R1) with 1.00 L 50% 2-BuOH/DMSO.
a. Charge 0.5V DMSO (0.5 L) followed by 0.5V 2-BuOH (0.5 L) to R1.

9. Polish filter the contents of R1 into R2.
a. Total volume at this point is approximately 4V (4 L).

10. Charge 1.50 L 2-BuOH to R2 (via a polish filter if GMP).

11. Charge sulfasalazine-DEA Form A seed (3.50 g) at <25° C. (i.e. r.t.).

12. Stir for 1-18 h.
a. Note: it will be difficult to see that solids are present. Transfer an aliquot to a 20 mL scintillation vial and look for solids using a flashlight.

13. Charge 15.50 L 2-BuOH over >5 hours.
a. Typical 2-BuOH addition time is 5-6 hours.
b. On the 700 g scale, the addition was done over 2 days where 5.7 V were added over 3h on one day and the remaining 9.8 V were added over 7h the following day.

14. Stir the slurry overnight. (>12h)
a. This is necessary to ensure the supernatant concentration is as low as it will go.

15. Measure the supernatant concentration: usually 7-8 mg/mL sulfasalazine free acid.

16. Filter the slurry.

17. Recirculate the mother liquors if necessary.

18. Displacement wash cake with 4.00 L 2-BuOH.

19. Dry solids until constant weight.
a. Oven drying at 45° C. over 4 days is fine.
b. Typical yield is 85% unadjusted yield.

HPLC (358 nm wavelength for sulfasalazine), NMR, XRPD and DVS analysis is performed to assess the purity and characterize the form of the product.

It was noted that other samples of sulfasalazine diethylamine salt (e.g., Form A) were yellow in color whereas the material prepared by the developed 2-butanol/DMSO process was orange. It was proposed that particle size can influence the color of the API, especially when the API is highly conjugated. A polarized light microscope image was obtained for both the SFS material and the material generated from the 2-butanol/DMSO process. It was concluded that the likely cause of the differences in colors of sulfasalazine diethylamine salt is particle size.

### Example 6

### Assessment of Stability of Sulfasalazine Diethylamine Salt

The diethylamine salt Form A of sulfasalazine was assessed for stability at 40° C./70% RH, by adapting the methods described herein. Packaging/Container Closer System: LDPE bag, double bagged with zip tie closure, single bag for each condition. See Tables 36-37. The compositions were assessed as indicated in Table 36. Impurities in the composition were analyzed using HPLC (Table 37).

TABLE 36
Stability Summary for diethylamine sulfasalazine salt at 40° C./75% RH

| Attribute | | Testing Interval (Months) | | |
|---|---|---|---|---|
| (Test Method) | Specification | T = 0 | 1 | 3 |
| Actual Pull Date | | Day 0 | Day 35 | Day 96 |
| Appearance | Report Results | Dark Orange | Dark Orange | Dark Orange |
| | | powder | powder | powder |
| Assay (Anhydrous, | Report Results | 99.6% (w/w %) | 97.9% (w/w %) | 96.5% (w/w %) |
| Solvent Free) | | | | |
| Total Impurities | Report Results | 0.73% | 0.87% | 0.77% |
| Water Content | Report Results | 0.4% (w/w %) | 1.1% (w/w %) | 1.1% (w/w %) |
| XRPD | Report Results | Conforms to | Conforms to | Conforms to |
| | | Form A | Form A | Form A |

TABLE 37
Summary of Individual Impurities in diethylamine sulfasalazine
salt at 40° C./75% RH during stability assessment

| Relative HPLC | |
|---|---|
| Retention Time | Testing Interval (Months) |

| (min) | Identity | T = 0 | 1 | 3 |
|---|---|---|---|---|
| 0.10 | ND | 0.03% | 0.17% | 0.06% |
| 0.57 | ND | 0.30% | 0.31% | 0.30% |
| 0.93 | ND | 0.08% | 0.08% | 0.10% |
| 1.14 | ND | 0.06% | 0.06% | 0.06% |
| 1.18 | ND | 0.16% | 0.17% | 0.17% |
| 1.32 | ND | 0.03% | ND | ND |
| 1.36 | ND | 0.07% | 0.08% | 0.08% |

ND = Not Detected or Not Determined

For comparison, the diethylamine salt form of sulfasalazine was also assessed for stability at 25° C./60% RH, by adapting the methods described herein. Packaging/Container Closer System: LDPE bag, double bagged with zip tie closure, single bag for each condition. See tables 36-37. The compositions were assessed as indicated in Table 38. Impurities in the composition were analyzed using HPLC (Table 39).

TABLE 38
Stability Summary for diethylamine sulfasalazine salt at 25° C./60% RH

| Attribute | | Testing Interval (Months) | | |
|---|---|---|---|---|
| (Test Method) | Specification | T = 0 | 1 | 3 |
| Actual Pull Date | | Day 0 | Day 35 | Day 96 |
| Appearance | Report Results | Dark Orange | Dark Orange | Dark Orange |
| | | powder | powder | powder |
| Assay (Anhydrous, | Report Results | 99.6% (w/w %) | 97.2% (w/w %) | 95.9% (w/w %) |
| Solvent Free) | | | | |
| Total Impurities | Report Results | 0.73% | 0.87% | 0.76% |
| Water Content | Report Results | 0.4% (w/w %) | 0.8% (w/w %) | 0.7% (w/w %) |
| XRPD | Report Results | Conforms to | Conforms to | Conforms to |
| | | Form A | Form A | Form A |

TABLE 39
Summary of Individual Impurities in diethylamine sulfasalazine
salt at 25° C./60% RH during stability assessment

| Relative HPLC | |
|---|---|
| Retention Time | Testing Interval (Months) |

| (min) | Identity | T = 0 | 1 | 3 |
|---|---|---|---|---|
| 0.10 | ND | 0.03% | 0.17% | 0.06% |
| 0.57 | ND | 0.30% | 0.31% | 0.29% |
| 0.93 | ND | 0.08% | 0.08% | 0.10% |
| 1.14 | ND | 0.06% | 0.06% | 0.06% |
| 1.18 | ND | 0.16% | 0.17% | 0.17% |
| 1.32 | ND | 0.03% | ND | ND |
| 1.36 | ND | 0.07% | 0.08% | 0.08% |

### Example 7

### Forced Degradation Studies of Exemplary Sulfasalazine Diethylamine Salt

The sulfasalazine diethylamine salt was subjected to the forced degradation conditions, including thermolytic, photolytic, oxidative and hydrolytic stresses. Target degradation for each condition was 5-20% impurities. After 14 days, if minimal degradation had occurred for a given condition, the drug substance was considered stable for that condition. Table 40 lists the experimental details of each stress condition.

TABLE 40
Forced Degradation Conditions

| Stress | | | | | |
|---|---|---|---|---|---|
| Condition | Duration | | Conditions | Control | Comments |
| Thermolytic | 8 | days | 85° C., | Unstressed | Solid state |
| | 14 | days | ambient | | |
| | | | humidity | | |
| Hydrolytic | 8 | days | 55° C./75% RH | Unstressed | Solid state |
| (humidity) | 14 | days | | | |
| Photolytic | 2X | ICH | Photochamber, | Dark control in | Solid state |
| | 3X | ICH | 25° C. | photochamber | |
| Hydrolytic, | 8 | days | 1N HCl, 55° C. | water, 55° C. | Slurry |
| acid | 14 | days | | | |
| Hydrolytic, | 8 | days | 1N NaOH, 55° C. | water, 55° C. | Slurry |
| base | 14 | days | | | |
| Oxidative | 8 | days | 3% H₂O₂, | Water, ambient | Slurry |
| | 14 | days | ambient | temperature | |
| | | | temperature | | |

The following general forced degradation methods were utilized to assess the sulfasalazine diethylamine salt.

Thermolytic. Drug substance was exposed to a controlled temperature of 80° C. for 14 days. Solid drug substance left at ambient temperature was used as a control sample. At appropriate intervals, sample solutions were prepared at the nominal concentration in diluent and analyzed. Minimal degradation was observed after 14 days.

Hydrolytic (humidity). Drug substance was exposed to a 75% RH atmosphere and a temperature of 55° C. Solid drug substance left at ambient temperature and humidity was used as a control sample. At appropriate intervals, sample solutions were prepared at the nominal concentration in diluent and analyzed. Minimal degradation was observed after 14 days.

Photolytic. Per ICH Q1B option 2 (Stability Testing: Photostability Testing of new Drug Substance and Products), drug substance was exposed to 2× and 3×ICH levels of UV and cool white fluorescent light. A control sample was prepared by wrapping a sample of solid drug substance in aluminum foil to block all light, then placing the sample in the photostability chamber along with the exposed sample. At appropriate intervals, sample solutions were prepared at the nominal concentration in diluent and analyzed. Minimal degradation was observed after 3× exposure.

Hydrolytic, Acid. Drug substance was prepared at approximately 4× the nominal sample concentration in 1 N HCl, then exposed to a temperature of 55° C. The sample remained as a slurry during the stress conditions. At appropriate intervals, an aliquot of the sample solution was neutralized 1:1, then diluted 1:1 with diluent and analyzed. A drug substance control sample was prepared in water and exposed to a temperature of 55° C. along with the stressed sample. A control sample solution was analyzed each day that a stressed sample solution was analyzed Minimal degradation was observed after 14 days.

Hydrolytic, Base. Drug substance was prepared at approximately 2× the nominal sample concentration in 1 N NaOH, then exposed to a temperature of 55° C. The sample remained in solution during the stress conditions. At appropriate intervals, an aliquot of the sample solution was neutralized 1:1, then diluted 1:1 with diluent and analyzed. A drug substance control sample was prepared in water and exposed to a temperature of 55° C. along with the stressed sample. A control sample solution was analyzed each day that a stressed sample solution was analyzed. Minimal degradation was observed after 14 days.

Oxidative. Drug substance was prepared at approximately 4× the nominal sample concentration in 3% H₂O₂. At appropriate intervals, an aliquot of the sample solution was diluted 1:4 with diluent and analyzed. A drug substance control sample was prepared in water and kept in dark, ambient conditions. A control sample solution was analyzed each day that a stressed sample was analyzed. Approximately 1.6% degradation was observed after 14 days.

Solid Control. The “solid control” represents completely unstressed drug substance. The solid control was prepared in diluent and analyzed immediately. The solid control was used to establish an initial purity value. This value was used to calculate the percent change values for the thermolytic and hydrolytic (humidity) studies, as well as the percent change for the photolytic and solution control samples.

The results of the forced degradation study are reported in Table 41.

TABLE 41
Forced Degradation Results

| | | | Area % | % | Peak |
|---|---|---|---|---|---|
| Condition | Duration | | (main band) | Change | Purity |
| Solid Control | 0 | days | 99.08 | N/A | N/A |
| Thermolytic | 8 | days | 98.96 | 0.12 | N/A |
| | 14 | days | 99.03 | 0.05 | Pass |
| Hydrolytic | 8 | days | 98.97 | 0.11 | N/A |
| (humidity) | 14 | days | 99.10 | −0.02 | Pass |
| Solution Control | 8 | days | 98.88 | 0.20 | N/A |
| (water, ambient) | 14 | days | 98.88 | 0.20 | N/A |
| Oxidative | 8 | days | 97.52 | 1.36 | N/A |
| | 14 | days | 97.30 | 1.58 | Pass |
| Solution Control | 8 | days | 98.84 | 0.24 | N/A |
| (water, 55° C.) | 14 | days | 98.69 | 0.39 | N/A |
| Hydrolytic, | 8 | days | 99.03 | −0.19 | N/A |
| Acid | 14 | days | 98.93 | −0.25 | Pass |
| Hydrolytic, | 8 | days | 99.03 | −0.19 | N/A |
| Base | 14 | days | 98.93 | −0.25 | Pass |
| Photolytic | 2X | ICH | 99.13 | −0.05 | N/A |
| Control | 3X | ICH | 99.13 | −0.05 | N/A |
| Photolytic | 2X | ICH | 98.71 | 0.42 | N/A |
| | 3X | ICH | 99.03 | 0.10 | Pass |

In the solid state form, the sulfasalazine diethylamine salt substance demonstrates reasonable stability to thermolytic, hydrolytic (humidity) and photolytic stresses. The drug substance is stable under strong acidic and basic conditions. There is measurable degradation for the oxidative stress after 8 days exposure. The peak purity values for the sulfasalazine diethylamine salt substance gives passing results for all of the final stress conditions.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A water-soluble crystalline salt of sulfasalazine.

2. The crystalline salt of claim 1, wherein the crystalline salt:
is substantially non-hygroscopic;
has a solubility of 1 mg/mL or more in an aqueous buffer at about pH 7 and 25° C.;
is polymorphically stable; and/or
is storage stable.

3. The crystalline salt of claim 1, wherein the crystalline salt is a pharmaceutically acceptable basic salt of sulfasalazine and an acid.

4. The crystalline salt of claim 3, wherein the acid is an organic sulfonic acid.

5. The crystalline salt of claim 3, wherein the acid is selected from benzenesulfonic acid, ethanedisulfonic acid, ethane sulfonic acid, methane sulfonic acid, naphthalene-1,5-disulfonic acid, p-toluenesulfonic acid and sulfuric acid.

6. The crystalline salt of claim 1, wherein the crystalline salt is a pharmaceutically acceptable acid salt of sulfasalazine and an organic amine base.

7. The crystalline salt of claim 6, wherein the base is selected from diethylamine, L-lysine, triethanolamine, tromethamine, piperazine, benzathine, diethanolamine and L-arginine.

8. The crystalline salt of claim 7, wherein the crystalline salt is crystalline sulfasalazine diethylamine (1:1) salt.

9. The crystalline salt of claim 8, wherein the crystalline salt is characterized by the X-ray Powder Diffraction Pattern as shown in FIG. 4, or is characterized by having a differential scanning calorimetry plot comprising one endothermic event with an onset temperature of about 191° C. when heated from about 25° C. to about 300° C.

10. The crystalline salt of claim 7, wherein the crystalline salt is crystalline sulfasalazine tromethamine (1:1) salt.

11. The crystalline salt of claim 10, wherein the crystalline salt is characterized by the X-ray Powder Diffraction Pattern as shown in FIG. 7, or is characterized by having a differential scanning calorimetry plot comprising endothermic events with an onset temperature of about 67° C. and about 123° C., when heated from about 25° C. to about 300° C.

12. A pharmaceutical composition comprising the crystalline salt of claim 1 and a pharmaceutical acceptable carrier, diluent or excipient.

13. A method of treating disease or condition that is a neurological related disease, a neurodegenerative disease, an inflammatory disease or condition or cancer, the method comprising administering to a subject in need thereof a therapeutically effective amount of a crystalline salt of claim 1.

14. The method of claim 13, wherein the disease or condition is:
a) a neurological related disease that is epilepsy;
b) an epilepsy selected from Dravet syndrome, Lennox-Gastaut syndrome, Doose syndrome, West syndrome, Angelman Syndrome, Benign Rolandic Epilepsy, CDKL5 Disorder, Childhood and Juvenile Absence Epilepsy, Doose Syndrome, Dravet Syndrome, Epilepsy with Myoclonic-Absences, Glut 1 Deficiency Syndrome, Infantile Spasms and West's Syndrome, Juvenile Myoclonic Epilepsy, Lafora Progressive Myoclonus Epilepsy, Landau-Kleffner Syndrome, Lennox-Gastaut Syndrome, Ohtahara Syndrome, Panayiotopoulos Syndrome, PCDH19 Epilepsy, Rasmussen's Syndrome, Ring Chromosome 20 Syndrome, Reflex Epilepsies, TBCK-related ID Syndrome, Hypothalamic Hamartoma, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Focal Cortical Dysplasia and epileptic encephalopathies. In another aspect of the method, the seizure disease or disorder is selected from the group consisting of Childhood and Juvenile Absence Epilepsy, Infantile Spasms and West's Syndrome, Juvenile Myoclonic Epilepsy, Frontal Lobe Epilepsy, Epilepsy with Generalized Tonic-Clonic Seizures Alone, Progressive Myoclonic Epilepsies, Temporal Lobe Epilepsy, Tuberous Sclerosis Complex, Rasmussen's Syndrome, Hypothalamic Hamartoma, Focal Cortical Dysplasia, epileptic encephalopathies, and Long-term epilepsy associated tumors (LEATs) for example ganglioglioma, oligodendroglioma, and dysembryoplastic neuroepithelial tumors (DNETs);
c) a neurodegenerative disease selected from Alexander disease, Alzheimer's disease (AD), frontotemporal dementia, HIV-associated dementia and other dementias, amyotrophic lateral sclerosis, epilepsy, Huntington's disease (HD), ischemic stroke, Motor neurone diseases (MND), neuropathic pain, Parkinson's disease (PD) and PD-related disorders, Prion disease, Rett syndrome, Spinal muscular atrophy (SMA), Spinocerebellar ataxia (SCA), traumatic brain injury, tuberous sclerosis, progressive multiple sclerosis (P-MS), amyotrophic lateral sclerosis (ALS) and neuropathic pain;
d) an inflammatory disease or condition selected from inflammatory bowel diseases, ulcerative colitis, Crohn's disease, inflammatory arthritis diseases, ankylosing spondylitis, rheumatoid arthritis and psoriatic arthritis; or
e) a cancer selected from glial tumors, glioblastoma, lymphoma and pancreatic cancer.

15. The method of claim 14, wherein the disease or condition is refractory epilepsy.

16. The method of claim 13, wherein the subject is diagnosed as having intractable seizures.

17. The method of claim 13, wherein the crystalline salt is administered at a dosage and/or frequency effective to reduce the occurrence of side effects of sulfasalazine.

18. The method of claim 13, further comprising co-administering to the subject an antiepileptic agent or an ABCG2 inhibitor.

19. A method of preparing a crystalline sulfasalazine salt, the method comprising:
a) combining sulfasalazine and an organic amine base in an organic solvent under conditions sufficient to crystallize a sulfasalazine salt; and
b) isolating the sulfasalazine salt;
wherein the organic amine base is selected from diethylamine, L-lysine, triethanolamine, tromethamine, piperazine, benzathine, diethanolamine and L-arginine.

20. The method of claim 19, wherein:
a) the organic amine base is diethylamine and the solvent is ethanol
b) the organic amine base is L-lysine and the solvent is acetone;
c) the organic amine base is triethanolamine and the solvent is acetone; or
d) the organic amine base is tromethamine and the solvent is ethanol.
